(19) United States
(12) Patent Application Publication
(10) Pub. No.: US 2014/0275250 A1
(43) Pub. Date: Sep. 18, 2014

(54) Methods of Administering Monomethyl Fumarate

(71) Applicant: XenoPort, Inc., Santa Clara, CA (US)

(72) Inventor: Kenneth C. Cundy, Atherton, CA (US)

(73) Assignee: XenoPort, Inc., Santa Clara, CA (US)

(21) Appl. No.: 13/973,780

(22) Filed: Aug. 22, 2013

Related U.S. Application Data
(60) Provisional application No. 61/801,248, filed on Mar. 15, 2013.
(60) Provisional application No. 61/804,614, filed on Mar. 22, 2013.

(51) Int. Cl. A61K 31/225

(52) U.S. Cl. CPC A61K 31/225
USPC 514/547

(57) ABSTRACT

Methods of therapeutic treatment using monomethyl fumarate are disclosed.

### CROSS REFERENCE TO RELATED APPLICATIONS

This application claims the benefit under 35 U.S.C. §119(e) of U.S. Provisional Application Ser. Nos. 61/801,248, filed Mar. 15, 2013 and 61/804,614, filed Mar. 22, 2013, the contents of both of which are incorporated herein by reference in their entirety.

### TECHNICAL FIELD

Disclosed herein are therapeutic methods of treating diseases such as multiple sclerosis and psoriasis involving administration of monomethyl fumarate.

### BACKGROUND

Fumaric acid esters (FAEs) are approved in Germany for the treatment of psoriasis, are being evaluated in the United States for the treatment of psoriasis and multiple sclerosis, and have been proposed for use in treating a wide range of immunological, autoimmune, and inflammatory diseases and conditions.

FAEs and other fumaric acid derivatives have been proposed for use in treating a wide-variety of diseases and conditions involving immunological, autoimmune, and/or inflammatory processes including psoriasis (Joshi and Strebel, WO 1999/49858; U.S. Pat. No. 6,277,882; Mrowietz and Asadullah, Trends Mol Med 2005, 111(1), 43-48; and Yazdi and Mrowietz, Clinics Dermatology 2008, 26, 522-526); asthma and chronic obstructive pulmonary diseases (Joshi et al., WO 2005/023241 and US 2007/0027076); cardiac insufficiency including left ventricular insufficiency, myocardial infarction and angina pectoris (Joshi et al., WO 2005/023241; Joshi et al., US 2007/0027076); mitochondrial and neurodegenerative diseases such as Parkinson's disease, Alzheimer's disease, Huntington's disease, retinopathia pigmentosa and mitochondrial encephalomyopathy (Joshi and Strebel, WO 2002/055063, US 2006/0205659, U.S. Pat. No. 6,509,376, U.S. Pat. No. 6,858,750, and U.S. Pat. No. 7,157,423); transplantation (Joshi and Strebel, WO 2002/055063, US 2006/0205659, U.S. Pat. No. 6,359,003, U.S. Pat. No. 6,509,376, and U.S. Pat. No. 7,157,423; and Lehmann et al., Arch Dermatol Res 2002, 294, 399-404); autoimmune diseases (Joshi and Strebel, WO 2002/055063, U.S. Pat. No. 6,509,376, U.S. Pat. No. 7,157,423, and US 2006/0205659) including multiple sclerosis (MS) (Joshi and Strebel, WO 1998/52549 and U.S. Pat. No. 6,436,992; Went and Lieberburg, US 2008/0089896; Schimrigk et al., Eur J Neurology 2006, 13, 604-610; and Schilling et al., Clin Experimental Immunology 2006, 145, 101-107); ischemia and reperfusion injury (Joshi et al., US 2007/0027076); AGE-induced genome damage (Heidland, WO 2005/027899); inflammatory bowel diseases such as Crohn's disease and ulcerative colitis; arthritis; and others (Nilsson et al., WO 2006/037342 and Nilsson and Muller, WO 2007/042034).

Fumaderm®, an enteric coated tablet containing a mixture of salts of monoethyl fumarate and dimethyl fumarate (DMF) was approved in Germany in 1994 for the treatment of psoriasis. Fumaderm® is dosed three times per day with 1-2 grams/day administered for the treatment of psoriasis.

Metabolites of DMF in human urine are reported by Rosami et al., in Journal of Investigative Dermatology (2009) 129, 231-234. The authors have reported the results of an in vivo study where urine samples of psoriasis patients were analyzed for mercapturic acids of MMF, MEF, and DMF [mixture of N-acetyl-S-(1-carboxy-2-methoxycarbonylethyl)cysteine and N-acetyl-S-(2-carboxy-1-methoxycarbonylethyl)cysteine) (NAC-MMS), mixture of N-acetyl-S-(1-carboxy-2-methoxycarbonylethyl)cysteine and N-acetyl-S-(2-carboxy-1-ethoxycarbonylethyl)cysteine (NAC-MES), and N-acetyl-S-(1,2-dimethoxycarbonylethyl)cysteine (NAC-DMS)] after oral intake of Fumaderm under fasting conditions. According to the authors, MMF-GSH (monomethylfumarate-glutathione adduct or MMF-GA) does not represent a significant metabolite of DMF, and that urinary NAC-MMS derives from degradation of DMF-GSH (dimethylfumarate-glutathione adduct). The authors have not reported blood metabolites. In addition, Joshi et al., disclose composition and preparation of DMF-GSH (dimethylfumarate-glutathione adduct) and analogs in U.S. Pat. No. 8,067,467.

Biogen Idec's BG-12 product, a delayed release (i.e., enteric coated microtablets) oral dosage form of the dimethyl fumarate, has been in clinical testing for the treatment of multiple sclerosis. Details concerning the clinical testing of BG-12 are disclosed in Sheikh et al., Safety Tolerability and Pharmacokinetics of BG-12 Administered with and without Aspirin, Key Findings from a Randomized, Double-blind, Placebo-controlled Trial in Healthy Volunteers, Poster PO4.136 presented at the 64^{th} Annual Meeting of the American Academy of Neurology, Apr. 21-28, 2012, New Orleans, La.; Dawson et al., Bioequivalence of BG-12 (Dimethyl Fumarate) Administered as a Single 240 mg Capsule and Two 120 mg Capsules: Findings from a Randomized, Two-period Crossover Study, Poster P913 presented at the 28th Congress of the European Committee for Treatment and Research in Multiple Sclerosis, Oct. 10-13, 2012, Lyon, France; and Woodworth et al., Pharmacokinetics of Oral BG-12 Alone Compared with BG-12 and Interferon β-1a or Glatiramer Acetate Administered Together, Studied in Health Volunteers, Poster PO4.207 presented at the 62^{nd} Annual Meeting of the American Academy of Neurology, Apr. 10-17, 2010, Toronto, Ontario, Canada, Placebo-controlled phase 3 study of oral BG-12 for relapsing multiple sclerosis, Gold R, et al., N Engl J. Med. 2012 Sep. 20; 367(12):1098-107, Erratum in: N Engl J. Med. 2012 Dec. 13; 367(24):2362, Placebo-controlled phase 3 study of oral BG-12 or glatiramer in multiple sclerosis, Fox R J, et al, N Engl J. Med. 2012 Sep. 20; 367(12):1087-97. Erratum in: N Engl J. Med. 2012 Oct. 25; 367(17):1673.

Fumaric acid derivatives (Joshi and Strebel, WO 2002/055063, US 2006/0205659, and U.S. Pat. No. 7,157,423 (amide compounds and protein-fumarate conjugates); Joshi et al., WO 2002/055066 and Joshi and Strebel, U.S. Pat. No. 6,355,676 (mono and dialkyl esters); Joshi and Strebel, WO 2003/087174 (carbocyclic and oxacarbocylic compounds); Joshi et al., WO 2006/122652 (thiosuccinates); Joshi et al., US 2008/0233185 (dialkyl and diaryl esters); Nielsen and Bundgaard, J Pharm Sci 1988, 77(4), 285-298 (glycolamide ester prodrugs); and Nilsson et al., US 2008/0004344 (salts)) have been developed in an effort to overcome the deficiencies of current FAE therapy. Controlled release pharmaceutical compositions comprising fumaric acid esters are disclosed by Nilsson and Müller, WO 2007/042034; by Nilsson and Rupp, US 2012/0034274 and US 2012/0034303.

### SUMMARY

Disclosed herein are methods of administering a therapeutically effective amount of monomethyl fumarate (MMF) to treat a disease in a patient in need of such treatment. In a first aspect, the methods comprise administering MMF to a patient, at a dose and dosing frequency that achieves (i) a total area under a curve plotting average molar concentration of monomethyl fumarate-glutathione adducts in the blood plasma of the patient versus time (AUC_{molar-MMF-GA}); and (ii) a total area under a curve plotting average molar concentration of monomethyl fumarate in the blood plasma of the patient versus time (AUC_{molar-MMF}); wherein the ratio of AUC_{molar-MMF-GA}:AUC_{molar-MMF} is greater than 2%.

In some embodiments, the ratio of AUC_{molar-MMF-GA}:AUC_{molar-MMF} is greater than 4%.

In some embodiments, the ratio of AUC_{molar-MMF-GA}:AUC_{molar-MMF} is from 4% to 100%.

In some embodiments, the ratio of AUC_{molar-MMF-GA}:AUC_{molar-MMF} is from 5% to 50%.

In some embodiments, the ratio of AUC_{molar-MMF-GA}:AUC_{molar-MMF} is from 5% to 20%.

In some embodiments, the ratio of AUC_{molar-MMF-GA}:AUC_{molar-MMF} is from 20% to 35%.

In some embodiments, the ratio of AUC_{molar-MMF-GA}:AUC_{molar-MMF} is from 35% to 50%.

In some embodiments, the administration is systemic. In some embodiments, the administration is oral.

In a second aspect, the methods comprise first selecting an MMF dose and dosing frequency that achieves similar AUC_{molar-MMF-GA} and AUC_{molar-MMF} values as mentioned above, but as mean values over a population of patients, and then treating individual patient(s) using that selected MMF dose and dosing frequency. In some embodiments, MMF is first administered to a population of patients at a plurality of different combinations of MMF dose and dosing frequency. One of the administered combinations is chosen on the basis that said combination achieves (i) a mean total area under a curve plotting average molar concentration of monomethyl fumarate-glutathione adducts in blood plasma of the population of patients versus time (mean AUC_{molar-MMF-GA}); (ii) a mean total area under a curve plotting average molar concentration of monomethyl fumarate in the blood plasma of the population of patients versus time (mean AUC_{molar-MMF}), wherein the ratio of mean AUC_{molar-MMF-GA}:mean AUC_{molar-MMF} is greater than 2%. Thereafter, an individual patient in need of such treatment is administered monomethyl fumarate at said at least one combination of MMF dose and dosing frequency.

In some embodiments, the ratio of mean AUC_{molar-MMF-GA}:mean AUC_{molar-MMF} is greater than 4%.

In some embodiments, the ratio of mean AUC_{molar-MMF-GA}:mean AUC_{molar-MMF} is from 4% to 100%.

In some embodiments, the ratio of mean AUC_{molar-MMF-GA}:mean AUC_{molar-MMF} is from 5% to 50%.

In some embodiments, the ratio of mean AUC_{molar-MMF-GA}:mean AUC_{molar-MMF} is from 5% to 20%.

In some embodiments, the ratio of mean AUC_{molar-MMF-GA}:mean AUC_{molar-MMF} is from 20% to 35%.

In some embodiments, the ratio of mean AUC_{molar-MMF-GA}:mean AUC_{molar-MMF} is from 35% to 50%.

In a third aspect, the present disclosure provides methods of administering a therapeutically effective amount of monomethyl fumarate to treat a disease in each patient of a population of patients in need of such treatment, comprising administering the monomethyl fumarate to each patient at a monomethyl fumarate dose and dosing frequency that achieves formation of MMF-GA (monomethyl fumarate-glutathione adducts) in blood plasma. In some embodiments, the mean maximum concentration (C_{max-MMF-GA}) of MMF-GA is at least 2% of the mean maximum concentration (C_{max-MMF}) of MMF.

In some embodiments, the mean maximum concentration (C_{max-MMF-GA}) of MMF-GA is at least 4% of the mean maximum concentration (C_{max-MMF}) of MMF.

In some embodiments, the mean maximum concentration (C_{max-MMF-GA}) of MMF-GA is from 4% to 50% of the mean maximum concentration (C_{max-MMF}) of MMF.

In some embodiments, the mean maximum concentration (C_{max-MMF-GA}) of MMF-GA is from 5% to 50% of the mean maximum concentration (C_{max-MMF}) of MMF.

In some embodiments, the mean maximum concentration (C_{max-MMF-GA}) of MMF-GA is from 5% to 20% of the mean maximum concentration (C_{max-MMF}) of MMF.

In some embodiments, the mean maximum concentration (C_{max-MMF-GA}) of MMF-GA is from 20% to 35% of the mean maximum concentration (C_{max-MMF}) of MMF.

In some embodiments, the mean maximum concentration (C_{max-MMF-GA}) of MMF-GA is from 35% to 50% of the mean maximum concentration (C_{max-MMF}) of MMF.

In a fourth aspect, the present disclosure provides methods of administering a therapeutically effective amount of monomethyl fumarate to treat a disease in a patient in need of such treatment, comprising administering the monomethyl fumarate to the patient at a monomethyl fumarate dose and dosing frequency that achieves formation of MMF-GA (monomethyl fumarate-glutathione adducts) in blood plasma. In some embodiments, the maximum concentration (C_{max-MMF-GA}) of MMF-GA is at least 2% of the maximum concentration (C_{max-MMF}) of MMF.

In some embodiments, the maximum concentration (C_{max-MMF-GA}) of MMF-GA is at least 4% of the maximum concentration (C_{max-MMF}) of MMF.

In some embodiments, the maximum concentration (C_{max-MMF-GA}) of MMF-GA is from 4% to 50% of the maximum concentration (C_{max-MMF}) of MMF.

In some embodiments, the maximum concentration (C_{max-MMF-GA}) of MMF-GA is from 5% to 50% of the maximum concentration (C_{max-MMF}) of MMF.

In some embodiments, the maximum concentration (C_{max-MMF-GA}) of MMF-GA is from 5% to 20% of the maximum concentration (C_{max-MMF}) of MMF.

In some embodiments, the maximum concentration (C_{max-MMF-GA}) of MMF-GA is from 20% to 35% of the maximum concentration (C_{max-MMF}) of MMF.

In some embodiments, the maximum concentration (C_{max-MMF-GA}) of MMF-GA is from 35% to 50% of the maximum concentration (C_{max-MMF}) of MMF.

In some embodiments, the monomethyl fumarate-glutathione adducts are chosen from:

[chemical structure]

and diastereomers thereof.

In some embodiments, the monomethyl fumarate-glutathione adducts are chosen from:

[chemical structure]

diastereomers thereof, and salts of any of the foregoing.

In some embodiments, the monomethyl fumarate-glutathione adducts are chosen from:

[chemical structure]

and diastereomers thereof.

In some embodiments, the monomethyl fumarate-glutathione adducts are chosen from:

[chemical structure]

diastereomers thereof, and salts of any of the foregoing.

In some embodiments, the monomethyl fumarate-glutathione adducts are in ionic forms.

In some embodiments, the monomethyl fumarate-glutathione adducts are in zwitterionic forms.

In some embodiments, the monomethyl fumarate-glutathione adducts are chosen from:

[chemical structure]

diastereomers thereof, and salts of any of the foregoing.

In some embodiments, the monomethyl fumarate-glutathione adducts are chosen from:

[chemical structure]

diastereomers thereof, and salts of any of the foregoing.

In some embodiments, the monomethyl fumarate-glutathione adduct is in any form, which is a result of a physiological transformation. In vivo, the monomethyl fumarate-glutathione adduct may be in a form other than the ionic or non-ionic forms shown in the chemical structures disclosed herein, and may be, for example, in a salt form. Thus, the monomethyl fumarate-glutathione adduct may be in the form of any naturally occurring physiological salt. For example, the monomethyl fumarate-glutathione adduct may be an HCl salt or a phosphate salt.

In some embodiments, the monomethyl fumarate is administered to the patient at a dose of from 50 to 1000 mg monomethyl fumarate per day. In some embodiments, the patient is dosed at a total dose of about 100 to 900 mg per day. In some embodiments, the patient is dosed at a total dose of about 200 to 800 mg per day. In some embodiments, the patient is dosed at a total dose of about 200 to 700 mg per day. In some embodiments, the patient is dosed at a total dose of about 200 to 600 mg per day.

In some embodiments, the monomethyl fumarate is administered to the patient at a dose of from 50 to 600 mg monomethyl fumarate per day.

In some embodiments, the monomethyl fumarate is administered to the patient at a dose of from 100 to 600 mg monomethyl fumarate per day.

In some embodiments, the monomethyl fumarate is administered to the patient at a dose of from 300 to 600 mg monomethyl fumarate per day.

In some embodiments, the monomethyl fumarate is administered to the patient at a dosing frequency of from once per day to three times per day.

In some embodiments, the disease is chosen from multiple sclerosis and psoriasis.

In some embodiments, the MMF-GA concentration in the blood plasma reaches the C_{max-MMF-GA} value within a time period of 2 to 10 hours after the administration.

In some embodiments, the MMF-GA concentration in the blood plasma reaches the C_{max-MMF-GA} value within a time period of 2 to 4 hours after the administration.

In some embodiments, the MMF-GA concentration in the blood plasma reaches the C_{max-MMF-GA} value within a time period of 3 to 4 hours after the administration.

In some embodiments, the MMF-GA concentration in the blood plasma reaches the C_{max-MMF-GA} value within a time period of 4 to 9 hours after the administration.

In some embodiments, the MMF-GA concentration in the blood plasma reaches the C_{max-MMF-GA} value within a time period of about 3 to 4 hours after the administration. In some embodiments the time period is about 4 to 5 hours. In some embodiments the time period is about 5 to 6 hours. In some embodiments the time period is about 6 to 7 hours. In some embodiments the time period is about 7 to 8 hours. In some embodiments the time period is about 8 to 9 hours.

In some embodiments, the patient is fasted. In some embodiments, the patient is fed.

In some embodiments, the administration is systemic administration.

In some embodiments, the administration is oral administration.

The therapeutic methods disclosed herein can be used to treat any number of diseases for which MMF and/or fumaric acid esters are known or thought to be therapeutically effective. In certain embodiments, the therapeutic treatments disclosed herein can be used to treat adrenal leukodystrophy, AGE-induced genome damage, Alexanders Disease, alopecia greata, Alper's Disease, Alzheimer's disease, amyotrophic lateral sclerosis, angina pectoris, arthritis, asthma, balo concentric sclerosis, Behcet's disease, bollus pemphigoid, Canavan disease, cardiac insufficiency including left ventricular insufficiency, central nervous system vasculitis, Charcott-Marie-Tooth Disease, childhood ataxia with central nervous system hypomyelination, chronic idiopathic peripheral neuropathy, chronic obstructive pulmonary disease, Crohn's disease, cutaneous lupus, dermatitis (contact, acute and chronic), diabetic retinopathy, graft versus host disease, granulomas, hepatitis C viral infection, herpes simplex viral infection, human immunodeficiency viral infection, Huntington's disease, irritable bowel disorder, ischemia, Krabbe Disease, lichen planus, macular degeneration, mitochondrial encephalomyopathy, monomelic amyotrophy, multiple sclerosis, myocardial infarction, neurodegeneration with brain iron accumulation, neuromyelitis optica, neurosarcoidosis, NF-κB mediated diseases, optic neuritis, pareneoplastic syndromes, Parkinson's disease, Pelizaeus-Merzbacher disease, pemphigus, primary lateral sclerosis, progressive supranuclear palsy, psoriasis, pyoderma gangrenosum, reperfusion injury, retinopathia pigmentosa, sarcoidosis, Schilders Disease, subacute necrotizing myelopathy, susac syndrome, transplantation rejection, transverse myelitis, a tumor, ulcerative colitis or Zellweger's syndrome. In some embodiments, the therapeutic treatments disclosed herein can be used for the treatment of multiple sclerosis and psoriasis.

### FIGURES

FIG. 1 shows the plasma molar concentrations of MMF and MMF-glutathione adducts following the oral dosing of enteric-coated sustained released tablet of Example 2 to fasted healthy adult patients.

FIG. 2 shows the plasma molar concentrations of MMF and MMF-glutathione adducts following the oral dosing of enteric-coated sustained released tablet of Example 2 to fed healthy adult patients.

FIG. 3 shows the plasma molar concentrations of MMF and MMF-glutathione adducts following the oral dosing of enteric-coated sustained released tablet of Example 4 to fasted healthy adult patients.

FIG. 4 shows the plasma molar concentrations of MMF and MMF-glutathione adducts following the oral dosing of enteric-coated sustained released tablet of Example 4 to fed healthy adult patients.

FIG. 5 shows the plasma molar concentrations of MMF and MMF-glutathione adducts following the oral dosing of non enteric-coated sustained released tablet of Example 8 to fasted healthy adult patients.

FIG. 6 shows the plasma molar concentrations of MMF and MMF-glutathione adducts following the oral dosing of non enteric-coated sustained released tablet of Example 8 to fed healthy adult patients.

FIG. 7 shows the plasma molar concentrations of MMF and MMF-glutathione adducts following the oral dosing of enteric-coated sustained released tablet of Example 14 to fasted healthy adult patients.

FIG. 8 shows the plasma molar concentrations of MMF and MMF-glutathione adducts following the oral dosing of enteric-coated sustained released tablet of Example 14 to fed healthy adult patients.

### DEFINITIONS

A dash (“-”) that is not between two letters or symbols is used to indicate a point of attachment for a moiety or substituent. For example, —CONH₂ is bonded through the carbon atom.

The terms “administering monomethyl fumarate” and “administration of monomethyl fumarate” as used herein include administration methods in which the monomethyl fumarate is directly administered to a patient, e.g., by putting the monomethyl fumarate directly into a dosage form that is administered to the patient, as well as methods in which the monomethyl fumarate is indirectly administered to a patient, e.g., by putting a precursor or prodrug of monomethyl fumarate, e.g., DMF, directly into a dosage form that is administered to the patient. Other prodrugs of monomethyl fumarate include compounds of Formulae (I) through (IV) of U.S. Provisional Patent Application Ser. No. 61/800,132, filed Mar. 15, 2013 entitled, “Methods of Administering Monomethyl Fumarate and Prodrugs Thereof having Reduced Side Effects”. The disclosures of these MMF prodrugs and methods for their synthesis are incorporated herein by reference. Of these, (N,N-diethylcarbamoyl)methyl methyl (2E)but-2-ene-1,4-dioate is the MMF source used in the Examples herein.

“Alkyl” refers to a saturated or unsaturated, branched, or straight-chain, monovalent hydrocarbon radical derived by the removal of one hydrogen atom from a single carbon atom of a parent alkane, alkene, or alkyne. Examples of alkyl groups include, but are not limited to, methyl; ethyls such as ethanyl, ethenyl, and ethynyl; propyls such as propan-1-yl, propan-2-yl, prop-1-en-1-yl, prop-1-en-2-yl, prop-2-en-1-yl (allyl), prop-1-yn-1-yl, prop-2-yn-1-yl, etc.; butyls such as butan-1-yl, butan-2-yl, 2-methyl-propan-1-yl, 2-methyl-propan-2-yl, but-1-en-1-yl, but-1-en-2-yl, 2-methyl-prop-1-en-1-yl, but-2-en-1-yl, but-2-en-2-yl, buta-1,3-dien-1-yl, buta-1,3-dien-2-yl, but-1-yn-1-yl, but-1-yn-3-yl, but-3-yn-1-yl, etc.; and the like.

The term “alkyl” is specifically intended to include groups having any degree or level of saturation, i.e., groups having exclusively single carbon-carbon bonds, groups having one or more double carbon-carbon bonds, groups having one or more triple carbon-carbon bonds, and groups having combinations of single, double, and triple carbon-carbon bonds. Where a specific level of saturation is intended, the terms alkanyl, alkenyl, and alkynyl are used. In certain embodiments, an alkyl group can have from 1 to 20 carbon atoms (C₁₋₂₀) in certain embodiments, from 1 to 10 carbon atoms (C₁₋₁₀), in certain embodiments from 1 to 8 carbon atoms (C₁₋₈), in certain embodiments, from 1 to 6 carbon atoms (C₁₋₆), in certain embodiments from 1 to 4 carbon atoms (C₁₋₄), and in certain embodiments, from 1 to 3 carbon atoms (C₁₋₃).

“Aryl” refers to a monovalent aromatic hydrocarbon radical derived by the removal of one hydrogen atom from a single carbon atom of a parent aromatic ring system. Aryl encompasses benzene; bicyclic ring systems wherein at least one ring is carbocyclic and aromatic, for example, naphthalene, indane, and tetralin; and tricyclic ring systems wherein at least one ring is carbocyclic and aromatic, for example, fluorene. Aryl encompasses multiple ring systems having at least one carbocyclic aromatic ring fused to at least one carbocyclic aromatic ring, cycloalkyl ring, or heterocycloalkyl ring. For example, aryl includes a phenyl ring fused to a 5- to 7-membered heterocycloalkyl ring containing one or more heteroatoms chosen from N, O, and S. For such fused, bicyclic ring systems wherein only one of the rings is a carbocyclic aromatic ring, the radical carbon atom may be at the carbocyclic aromatic ring or at the heterocycloalkyl ring. Examples of aryl groups include, but are not limited to, groups derived from aceanthrylene, acenaphthylene, acephenanthrylene, anthracene, azulene, benzene, chrysene, coronene, fluoranthene, fluorene, hexacene, hexaphene, hexylene, as-indacene, s-indacene, indane, indene, naphthalene, octacene, octaphene, octalene, ovalene, penta-2,4-diene, pentacene, pentalene, pentaphene, perylene, phenalene, phenanthrene, picene, pleiadene, pyrene, pyranthrene, rubicene, triphenylene, trinaphthalene, and the like. In certain embodiments, an aryl group can have from 6 to 20 carbon atoms (C₆₋₂₀), from 6 to 12 carbon atoms (C₆₋₁₂), from 6 to 10 carbon atoms (C₆₋₁₀), and in certain embodiments from 6 to 8 carbon atoms (C₆₋₈).

“Arylalkyl” refers to an acyclic alkyl radical in which one of the hydrogen atoms bonded to a carbon atom, typically a terminal or sp³ carbon atom, is replaced with an aryl group. Examples of arylalkyl groups include, but are not limited to, benzyl, 2-phenylethan-1-yl, 2-phenylethen-1-yl, naphthylmethyl, 2-naphthylethan-1-yl, 2-naphthylethen-1-yl, naphthobenzyl, 2-naphthophenylethan-1-yl and the like. Where specific alkyl moieties are intended, the nomenclature arylalkanyl, arylalkenyl, or arylalkynyl is used. In certain embodiments, an arylalkyl group is C₇₋₃₀ arylalkyl, e.g., the alkanyl, alkenyl or alkynyl moiety of the arylalkyl group is C₁₋₁₀ and the aryl moiety is C₆₋₂₀, in certain embodiments, an arylalkyl group is C₆₋₁₈ arylalkyl, e.g., the alkanyl, alkenyl or alkynyl moiety of the arylalkyl group is C₁₋₈ and the aryl moiety is C₆₋₁₀. In certain embodiments, an arylalkyl group is C₇₋₁₂ arylalkyl.

“AUC_{molar-MMF}” refers to the area under the plot of average molar concentration of monomethyl fumarate in the blood plasma of a patient versus time, during administration of monomethyl fumarate to the patient. The plot measurements are taken at multiple time points starting from 0.5 hours before a first dosing of a daily dosing regimen and for 24 hours following the first dosing.

“AUC_{molar-MMF-GA}” refers to the area under the plot of average molar concentration of monomethyl fumarate-glutathione adducts in the blood plasma of a patient versus time, during administration of monomethyl fumarate to the patient. The plot measurements are taken at multiple time points starting from 0.5 hours before a first dosing of a daily dosing regimen and for 24 hours following the first dosing.

“Mean AUC_{molar-MMF}” refers to the area under a plot of molar concentration of monomethyl fumarate from the blood plasmas of a population of patients, wherein each patient receives the same administration of monomethyl fumarate. The plot measurements are taken at multiple time points starting from 0.5 hours before a first dosing of a daily dosing regimen and for 24 hours following the first dosing. At each time point, the mean monomethyl fumarate concentration across the population of patients is selected, and the plot is drawn through those mean data points to obtain the curve under which the area is calculated.

“Mean AUC_{molar-MMF-GA}” refers to the area under a plot of molar concentration of monomethyl fumarate-glutathione adducts from the blood plasmas of a population of patients, wherein each patient receives the same administration of monomethyl fumarate. The plot measurements are taken at multiple time points starting from 0.5 hours before a first dosing of a daily dosing regimen and for 24 hours following the first dosing. At each time point, the mean monomethyl fumarate-glutathione adduct concentration across the population of patients is selected, and the plot is drawn through those mean data points to obtain the curve under which the area is calculated.

“C_{max-MMF}” refers to the maximum value of a MMF concentration versus time curve in blood plasma.

“C_{max-MMF-GA}” refers to the maximum value of a MMF-GA (monomethylfumarate-glutathione adducts) concentration versus time curve in blood plasma.

“Compounds” refers to chemical substances consisting of two or more different chemical elements that can be separated into simpler substances by chemical reactions. Compounds have a unique and defined chemical structure; they consist of a fixed ratio of atoms that are held together in a defined spatial arrangement by chemical bonds. Compounds include any specific compounds within a given chemical formula. Compounds may be identified either by their chemical structure and/or chemical name. Compounds are named using Chemistry 4-D Draw Pro, version 7.01c (ChemInnovation Software, Inc., San Diego, Calif.). When the chemical structure and chemical name conflict, the chemical structure is determinative of the identity of the compound. The compounds described herein may comprise one or more chiral centers and/or double bonds and therefore may exist as stereoisomers such as double-bond isomers (i.e., geometric isomers), enantiomers, or diastereomers. Accordingly, any chemical structures within the scope of the specification depicted, in whole or in part, with a relative configuration are deemed to encompass all possible enantiomers and stereoisomers of the illustrated compounds including the stereoisomerically pure form (e.g., geometrically pure, enantiomerically pure, or diastereomerically pure) and enantiomeric and stereoisomeric mixtures. Enantiomeric and stereoisomeric mixtures may be resolved into their component enantiomers or stereoisomers using separation techniques or chiral synthesis techniques well known to the skilled artisan. Compounds include, but are not limited to, optical isomers, racemates, and other mixtures. In such embodiments, a single enantiomer or diastereomer, i.e., optically active form can be obtained by asymmetric synthesis or by resolution of the racemates. Resolution of the racemates may be accomplished, for example, by conventional methods such as crystallization in the presence of a resolving agent, or chromatography using, for example, chiral stationary phases.

Compounds may also exist in several tautomeric forms including the enol form, the keto form, and mixtures thereof. Accordingly, the chemical structures depicted herein encompass all possible tautomeric forms of any illustrated compounds. Compounds also include isotopically labeled compounds where one or more atoms have an atomic mass different from the atomic mass conventionally found in nature. Examples of isotopes that may be incorporated into the compounds disclosed herein include, but are not limited to ²H, ³H, ¹¹C, ¹³C, ¹⁴C, ¹⁵N, ¹⁸O, ¹⁷O, etc. Compounds may exist in unsolvated forms as well as solvated forms, including hydrated forms and as N-oxides. In general, compounds as referred to herein may be free acid, hydrated, solvated, or N-oxides. Certain compounds may exist in multiple crystalline, co-crystalline, or amorphous forms. Compounds include pharmaceutically acceptable salts thereof, or pharmaceutically acceptable solvates of the free acid form of any of the foregoing, as well as crystalline forms of any of the foregoing.

Compounds also include solvates. A solvate refers to a molecular complex of a compound with one or more solvent molecules in a stoichiometric or non-stoichiometric amount. Such solvent molecules are those commonly used in the pharmaceutical art, which are known to be innocuous to a patient, e.g., water, ethanol, and the like. A molecular complex of a compound or moiety of a compound and a solvent can be stabilized by non-covalent intra-molecular forces such as, for example, electrostatic forces, van der Waals forces, or hydrogen bonds. The term “hydrate” refers to a solvate in which the one or more solvent molecules is water.

Further, when partial structures of compounds are illustrated, an asterisk (*) indicates the point of attachment of the partial structure to the rest of the molecule.

“Cycloalkyl” refers to a saturated or partially unsaturated cyclic alkyl radical. Where a specific level of saturation is intended, the nomenclature cycloalkanyl or cycloalkenyl is used. Examples of cycloalkyl groups include, but are not limited to, groups derived from cyclopropane, cyclobutane, cyclopentane, cyclohexane, and the like. In certain embodiments, a cycloalkyl group is C₃₋₁₅ cycloalkyl, C₃₋₁₂ cycloalkyl, and in certain embodiments, C₃₋₈ cycloalkyl.

“Cycloalkylalkyl” refers to an acyclic alkyl radical in which one of the hydrogen atoms bonded to a carbon atom, typically a terminal or sp³ carbon atom, is replaced with a cycloalkyl group. Where specific alkyl moieties are intended, the nomenclature cycloalkylalkanyl, cycloalkylalkenyl, or cycloalkylalkynyl is used. In certain embodiments, a cycloalkylalkyl group is C₄₋₃₀ cycloalkylalkyl, e.g., the alkanyl, alkenyl, or alkynyl moiety of the cycloalkylalkyl group is C₁₋₁₀ and the cycloalkyl moiety is C₃₋₂₀, and in certain embodiments, a cycloalkylalkyl group is C₃₋₂₀ cycloalkylalkyl, e.g., the alkanyl, alkenyl, or alkynyl moiety of the cycloalkylalkyl group is C₁₋₈ and the cycloalkyl moiety is C₃₋₁₂. In certain embodiments, a cycloalkylalkyl group is C₄₋₁₂ cycloalkylalkyl.

“Dimethyl fumarate” refers to the dimethyl ester of fumaric acid. The compound has a molecular weight of 144.13 daltons and the following chemical structure:

[chemical structure]

This compound is also known by the names Dimethyl (E)-butenedioate (IUPAC), trans-1,2-Ethylenedicarboxylic acid dimethyl ester and (E)-2-Butenedioic acid dimethyl ester. The compound is also referred to by the acronym DMF. DMF can be synthesized according to the methods described in Chinese Patent Publication CN 101318901A, the disclosures of which are incorporated herein by reference.

“Disease” refers to a disease, disorder, condition, or symptom of any of the foregoing.

“Drug” as defined under 21 U.S.C. §321(g)(1) means (A) articles recognized in the official United States Pharmacopoeia, official Homeopathic Pharmacopoeia of the United States, or official National Formulary, or any supplement to any of them; and (B) articles intended for use in the diagnosis, cure, mitigation, treatment, or prevention of disease in man or other animals; and (C) articles (other than food) intended to affect the structure or any function of the body of man or other animals.

“Glutathione” and “GSH” each refer to a tripeptide with a gamma peptide linkage between the amine group of cysteine (which is attached by normal peptide linkage to a glycine) and the carboxyl group of the glutamate side-chain. Glutathione has the following chemical structure:

[chemical structure]

GSH is an antioxidant, preventing damage to important cellular components caused by reactive oxygen species such as free radicals and peroxides. Glutathione has multiple functions in the body. It is the major endogenous antioxidant produced by the cells, participating directly in the neutralization of free radicals and reactive oxygen compounds, as well as maintaining exogenous antioxidants such as vitamins C and E in their reduced (active) forms. It helps regulate the nitric oxide cycle, which is critical for life but can be problematic if unregulated. It is used in metabolic and biochemical reactions such as DNA synthesis and repair, protein synthesis, prostaglandin synthesis, amino acid transport, and enzyme activation. Thus, every system in the body can be affected by the state of the glutathione system, especially the immune system, the nervous system, the gastrointestinal system and the lungs. It has a vital function in iron metabolism. Yeast cells depleted of or containing toxic levels of GSH show an intense iron starvation-like response and impairment of the activity of extra-mitochondrial ISC enzymes, followed by death.

GSH is known as a substrate in both conjugation reactions and reduction reactions, catalyzed by glutathione S-transferase enzymes in cytosol, microsomes, and mitochondria. However, it is also capable of participating in non-enzymatic conjugation with some chemicals. For example, patients taking acetaminophen form a metabolite in vivo: N-acetyl-p-benzoquinone imine (NAPQI). NAPQI becomes toxic when GSH is depleted by an overdose of acetaminophen. In such cases, glutathione is an essential antidote to the overdose. Glutathione conjugates to NAPQI and helps to detoxify it. In this capacity, it protects cellular protein thiol groups, which would otherwise become covalently modified; when all GSH has been spent, NAPQI begins to react with the cellular proteins, killing the cells in the process. One widely used treatment for an overdose of acetaminophen is the administration of compounds such as N-acetyl-L-cysteine, which are utilized by the body in the de novo synthesis of GSH.

“Halogen” refers to a fluoro, chloro, bromo, or iodo group. In certain embodiments, halogen refers to a chloro group.

“Heteroalkyl” by itself or as part of another substituent refer to an alkyl group in which one or more of the carbon atoms (and certain associated hydrogen atoms) are independently replaced with the same or different heteroatomic groups. Examples of heteroatomic groups include, but are not limited to, —O—, —S—, —O—O—, —S—S—, —O—S—, —NR¹³, ═N—N═, —N═N—, —N═N—NR¹³, —PR¹³—, —P(O)₂—, —POR¹³—, —O—P(O)₂—, —SO—, —SO₂—, —Sn(R¹³)₂—, and the like, where each R¹³ is independently chosen from hydrogen, C₁₋₆ alkyl, substituted C₁₋₆ alkyl, C₆₋₁₂ aryl, substituted C₆₋₁₂ aryl, C₇₋₁₈ arylalkyl, substituted C₇₋₁₈ arylalkyl, C₃₋₇ cycloalkyl, substituted C₃₋₇ cycloalkyl, C₃₋₇ heterocycloalkyl, substituted C₃₋₇ heterocycloalkyl, C₁₋₆ heteroalkyl, substituted C₁₋₆ heteroalkyl, C₆₋₁₂heteroaryl, substituted C₆₋₁₂ heteroaryl, C₇₋₁₈ heteroarylalkyl, or substituted C₇₋₁₈ heteroarylalkyl. Reference to, for example, a C₁₋₆ heteroalkyl, means a C₁₋₆ alkyl group in which at least one of the carbon atoms (and certain associated hydrogen atoms) is replaced with a heteroatom. For example C₁₋₆ heteroalkyl includes groups having five carbon atoms and one heteroatom, groups having four carbon atoms and two heteroatoms, etc. In certain embodiments, each R¹³ is independently chosen from hydrogen and C₁₋₃ alkyl. In certain embodiments, a heteroatomic group is chosen from —O—, —S—, —NH—, —N(CH₃)—, and —SO₂—; and in certain embodiments, the heteroatomic group is —O—.

“Heteroaryl” refers to a monovalent heteroaromatic radical derived by the removal of one hydrogen atom from a single atom of a parent heteroaromatic ring system. Heteroaryl encompasses multiple ring systems having at least one heteroaromatic ring fused to at least one other ring, which can be aromatic or non-aromatic. For example, heteroaryl encompasses bicyclic rings in which one ring is heteroaromatic and the second ring is a heterocycloalkyl ring. For such fused, bicyclic heteroaryl ring systems wherein only one of the rings contains one or more heteroatoms, the radical carbon may be at the aromatic ring or at the heterocycloalkyl ring. In certain embodiments, when the total number of N, S, and O atoms in the heteroaryl group exceeds one, the heteroatoms are not adjacent to one another. In certain embodiments, the total number of heteroatoms in the heteroaryl group is not more than two.

Examples of heteroaryl groups include, but are not limited to, groups derived from acridine, arsindole, carbazole, β-carboline, chromane, chromene, cinnoline, furan, imidazole, indazole, indole, indoline, indolizine, isobenzofuran, isochromene, isoindole, isoindoline, isoquinoline, isothiazole, isoxazole, naphthyridine, oxadiazole, oxazole, perimidine, phenanthridine, phenanthroline, phenazine, phthalazine, pteridine, purine, pyran, pyrazine, pyrazole, pyridazine, pyridine, pyrimidine, pyrrole, pyrrolizine, quinazoline, quinoline, quinolizine, quinoxaline, tetrazole, thiadiazole, thiazole, thiophene, triazole, xanthene, thiazolidine, oxazolidine, and the like. In certain embodiments, a heteroaryl group is from 4- to 20-membered heteroaryl (C₄₋₂₀), and in certain embodiments from 4- to 12-membered heteroaryl (C₄₋₁₀). In certain embodiments, heteroaryl groups are those derived from thiophene, pyrrole, benzothiophene, benzofuran, indole, pyridine, quinoline, imidazole, oxazole, or pyrazine. For example, in certain embodiments, C₅ heteroaryl can be furyl, thienyl, pyrrolyl, imidazolyl, pyrazolyl, isothiazolyl, isoxazolyl.

“Heterocycloalkyl” refers to a saturated or unsaturated cyclic alkyl radical in which one or more carbon atoms (and certain associated hydrogen atoms) are independently replaced with the same or different heteroatom; or to a parent aromatic ring system in which one or more carbon atoms (and certain associated hydrogen atoms) are independently replaced with the same or different heteroatom such that the ring system no longer contains at least one aromatic ring. Examples of heteroatoms to replace the carbon atom(s) include, but are not limited to, N, P, O, S, Si, etc. Examples of heterocycloalkyl groups include, but are not limited to, groups derived from epoxides, azirines, thiiranes, imidazolidine, morpholine, piperazine, piperidine, pyrazolidine, pyrrolidine, quinuclidine, and the like. In certain embodiments, a heterocycloalkyl group is C₅₋₁₀ heterocycloalkyl, C₅₋₈ heterocycloalkyl, and in certain embodiments, C₅₋₆ heterocycloalkyl.

“Leaving group” has the meaning conventionally associated with it in synthetic organic chemistry, i.e., an atom or a group capable of being displaced by a nucleophile and includes halogen such as chloro, bromo, fluoro, and iodo, acyloxy(alkoxycarbonyl) such as acetoxy and benzoyloxy, aryloxycarbonyl, mesyloxy, tosyloxy, trifluoromethanesulfonyloxy, aryloxy such as 2,4-dinitrophenoxy, methoxy, N,O-dimethylhydroxylamino, p-nitrophenolate, imidazolyl, and the like.

“Monomethyl fumarate” refers to the monomethyl ester of fumaric acid. The compound has a molecular weight of 130.10 daltons and the following chemical formula:

[chemical structure]

The compound is also commonly referred to as 2(E)-Butenedioic acid 1-methyl ester; (2E)-4-Methoxy-4-oxobut-2-enoic acid; Fumaric acid hydrogen 1-methyl ester; (2E)-2-Butenedioic acid 1-methyl ester; (E)-2-Butenedioic acid monomethyl ester; Monomethyl trans-ethylene-1,2-dicarboxylate; and methyl hydrogen fumarate. The compound is also referred to herein and elsewhere by the acronyms MMF and/or MHF. MMF can be synthesized according to the methods described in Dymicky, Preparation of Monomethyl Fumarate, Organic Preparations and Procedures International: The New Journal for Organic Synthesis, Vol 14, Issue 4, 1983; and Spatz et al., J. Org. Chem., 1958, 23 (10), 1559-1560.

“Multiple sclerosis” also known as “disseminated sclerosis” or “encephalomyelitis disseminata”, and sometimes referred to by the acronym MS, is an inflammatory disease in which the fatty myelin sheaths around the axons of the brain and spinal cord are damaged, leading to demyelination and scarring as well as a broad spectrum of signs and symptoms. Disease onset usually occurs in young adults, and it is more common in women. It has a prevalence that ranges between 2 and 150 per 100,000.

MS affects the ability of nerve cells in the brain and spinal cord to communicate with each other effectively. Nerve cells communicate by sending electrical signals called action potentials down long fibers called axons, which are contained within an insulating substance called myelin. In MS, the body's own immune system attacks and damages the myelin. When myelin is lost, the axons can no longer effectively conduct signals. The name multiple sclerosis refers to scars (sclerae better known as plaques or lesions) particularly in the white matter of the brain and spinal cord, which is mainly composed of myelin. Although much is known about the mechanisms involved in the disease process, the cause remains unknown. Theories include genetics or infections. Different environmental risk factors have also been found.

Almost any neurological symptom can appear with the disease, and the disease often progresses to physical and cognitive disability. MS takes several forms, with new symptoms occurring either in discrete attacks (relapsing forms) or accumulating over time (progressive forms). Between attacks, symptoms may go away completely, but permanent neurological deficits often occur, especially as the disease advances.

“Parent aromatic ring system” refers to an unsaturated cyclic or polycyclic ring system having a conjugated π (pi) electron system. Included within the definition of “parent aromatic ring system” are fused ring systems in which one or more of the rings are aromatic and one or more of the rings are saturated or unsaturated, such as, for example, fluorene, indane, indene, phenalene, etc. Examples of parent aromatic ring systems include, but are not limited to, aceanthrylene, acenaphthylene, acephenanthrylene, anthracene, azulene, benzene, chrysene, coronene, fluoranthene, fluorene, hexacene, hexaphene, hexylene, as-indacene, s-indacene, indane, indene, naphthalene, octacene, octaphene, octalene, ovalene, penta-2,4-diene, pentacene, pentalene, pentaphene, perylene, phenalene, phenanthrene, picene, pleiadene, pyrene, pyranthrene, rubicene, triphenylene, trinaphthalene, and the like.

“Parent heteroaromatic ring system” refers to an aromatic ring system in which one or more carbon atoms (and any associated hydrogen atoms) are independently replaced with the same or different heteroatom in such a way as to maintain the continuous π-electron system characteristic of aromatic systems and a number of out-of-plane π-electrons corresponding to the Hückel rule (4n+2). Examples of heteroatoms to replace the carbon atoms include, but are not limited to, N, P, O, S, and Si, etc. Specifically included within the definition of “parent heteroaromatic ring systems” are fused ring systems in which one or more of the rings are aromatic and one or more of the rings are saturated or unsaturated, such as, for example, arsindole, benzodioxan, benzofuran, chromane, chromene, indole, indoline, xanthene, etc. Examples of parent heteroaromatic ring systems include, but are not limited to, arsindole, carbazole, β-carboline, chromane, chromene, cinnoline, furan, imidazole, indazole, indole, indoline, indolizine, isobenzofuran, isochromene, isoindole, isoindoline, isoquinoline, isothiazole, isoxazole, naphthyridine, oxadiazole, oxazole, perimidine, phenanthridine, phenanthroline, phenazine, phthalazine, pteridine, purine, pyran, pyrazine, pyrazole, pyridazine, pyridine, pyrimidine, pyrrole, pyrrolizine, quinazoline, quinoline, quinolizine, quinoxaline, tetrazole, thiadiazole, thiazole, thiophene, triazole, xanthene, thiazolidine, oxazolidine, and the like.

“Patient” refers to a mammal, for example, a human.

“Pharmaceutically acceptable” refers to approved or approvable by a regulatory agency of the Federal or a state government or listed in the U.S. Pharmacopoeia or other generally recognized pharmacopoeia for use in animals, and more particularly in humans.

“Pharmaceutically acceptable salt” refers to a salt of a compound, which possesses the desired pharmacological activity of the parent compound. Such salts include acid addition salts, formed with inorganic acids such as hydrochloric acid, hydrobromic acid, sulfuric acid, nitric acid, phosphoric acid, and the like; or formed with organic acids such as acetic acid, propionic acid, hexanoic acid, cyclopentanepropionic acid, glycolic acid, pyruvic acid, lactic acid, malonic acid, succinic acid, malic acid, maleic acid, fumaric acid, tartaric acid, citric acid, benzoic acid, 3-(4-hydroxybenzoyl)benzoic acid, cinnamic acid, mandelic acid, methanesulfonic acid, ethanesulfonic acid, 1,2-ethane-disulfonic acid, 2-hydroxyethanesulfonic acid, benzenesulfonic acid, 4-chlorobenzenesulfonic acid, 2-naphthalenesulfonic acid, 4-toluenesulfonic acid, camphorsulfonic acid, 4-methylbicyclo[2.2.2]-oct-2-ene-1-carboxylic acid, glucoheptonic acid, 3-phenylpropionic acid, trimethylacetic acid, tertiary butylacetic acid, lauryl sulfuric acid, gluconic acid, glutamic acid, hydroxynaphthoic acid, salicylic acid, stearic acid, muconic acid, and the like; and salts formed when an acidic proton present in the parent compound is replaced by a metal ion, e.g., an alkali metal ion, an alkaline earth ion, or an aluminum ion; or coordinates with an organic base such as ethanolamine, diethanolamine, triethanolamine, N-methylglucamine, and the like. In certain embodiments, a pharmaceutically acceptable salt is the hydrochloride salt. In certain embodiments, a pharmaceutically acceptable salt is the sodium salt.

“Pharmaceutically acceptable vehicle” refers to a pharmaceutically acceptable diluent, a pharmaceutically acceptable adjuvant, a pharmaceutically acceptable excipient, a pharmaceutically acceptable carrier, or a combination of any of the foregoing with which a compound provided by the present disclosure may be administered to a patient and which does not destroy the pharmacological activity thereof and which is non-toxic when administered in doses sufficient to provide a therapeutically effective amount of the compound.

“Pharmaceutical composition” refers to a therapeutically active compound and at least one pharmaceutically acceptable vehicle, with which the compound is administered to a patient.

“Psoriasis” is an immune-mediated disease that affects the skin. It is typically a lifelong condition. Psoriasis occurs when the immune system mistakes a normal skin cell for a pathogen, and sends out faulty signals that cause overproduction of new skin cells. There are five types of psoriasis: plaque, guttate, inverse, pustular, and erythrodermic. The most common form, plaque psoriasis, is commonly seen as red and white hues of scaly patches appearing on the top first layer of the epidermis (skin). Some patients, though, have no dermatological signs or symptoms. The name psoriasis is from the Greek word, meaning roughly “itching condition” (psora “itch”+-sis “action, condition”).

In plaque psoriasis, skin rapidly accumulates at these sites, which gives it a silvery-white appearance. Plaques frequently occur on the skin of the elbows and knees, but can affect any area, including the scalp, palms of hands and soles of feet, and genitals. In contrast to eczema, psoriasis is more likely to be found on the outer side of the joint.

The disorder is a chronic recurring condition that varies in severity from minor localized patches to complete body coverage. Fingernails and toenails are frequently affected (psoriatic nail dystrophy) and can be seen as an isolated sign. Psoriasis can also cause inflammation of the joints, which is known as psoriatic arthritis. Between 10% and 30% of all people with psoriasis also have psoriatic arthritis.^{[5][6]}

The cause of psoriasis is not fully understood, but it is believed to have a genetic component and local psoriatic changes can be triggered by an injury to the skin known as the Koebner phenomenon. Various environmental factors have been suggested as aggravating to psoriasis, including oxidative stress, stress, withdrawal of systemic corticosteroid, as well as other environmental factors, but few have shown statistical significance.

“Substituted” refers to a group in which one or more hydrogen atoms are independently replaced with the same or substituent group(s). In certain embodiments, each substituent group is independently chosen from halogen, —OH, —CN, —CF₃, ═O, —NO₂, benzyl, —C(O)NH₂, —R¹¹, —OR¹¹, —C(O)R¹¹, —COOR¹¹, and —NR¹¹₂ wherein each R¹¹ is independently chosen from hydrogen and C₁₋₄ alkyl. In certain embodiments, each substituent group is independently chosen from halogen, —OH, —CN, —CF₃, —NO₂, benzyl, —R¹¹, —OR¹¹, and —NR¹¹₂ wherein each R¹¹ is independently chosen from hydrogen and C₁₋₄ alkyl. In certain embodiments, each substituent group is independently chosen from halogen, —OH, —CN, —CF₃, ═O, —NO₂, benzyl, —C(O)NR¹¹₂, —R¹¹, —OR¹¹, —C(O)R¹¹, —COOR¹¹, and —NR¹¹₂ wherein each R¹¹ is independently chosen from hydrogen and C₁₋₄ alkyl. In certain embodiments, each substituent group is independently chosen from —OH, C₁₋₄ alkyl, and —NH₂.

“Systemic administration” and “systemically administering” shall each mean a route of administration of a compound into the circulatory system of a patient in a therapeutically effective amount. In some non-limiting embodiments, administration can take place via enteral administration (absorption of the medication through the gastrointestinal tract) or parenteral administration (generally injection, infusion, or implantation). These terms are in contrast with topical and other types of local administration where a therapeutically effective amount is not in the circulatory system.

“Treating” or “treatment” of any disease refers to reversing, alleviating, arresting, or ameliorating a disease or at least one of the clinical symptoms of a disease, reducing the risk of acquiring at least one of the clinical symptoms of a disease, inhibiting the progress of a disease or at least one of the clinical symptoms of the disease or reducing the risk of developing at least one of the clinical symptoms of a disease. “Treating” or “treatment” also refers to inhibiting the disease, either physically, (e.g., stabilization of a discernible symptom), physiologically, (e.g., stabilization of a physical parameter), or both, and to inhibiting at least one physical parameter that may or may not be discernible to the patient. In certain embodiments, “treating” or “treatment” refers to protecting against or delaying the onset of at least one or more symptoms of a disease in a patient.

“Therapeutically effective amount” refers to the amount of a compound that, when administered to a subject for treating a disease, or at least one of the clinical symptoms of a disease, is sufficient to affect such treatment of the disease or symptom thereof. The “therapeutically effective amount” may vary depending, for example, on the compound, the disease and/or symptoms of the disease, severity of the disease and/or symptoms of the disease or disorder, the age, weight, and/or health of the patient to be treated, and the judgment of the prescribing physician. An appropriate amount in any given instance may be ascertained by those skilled in the art or capable of determination by routine experimentation.

“Therapeutically effective dose” refers to a dose that provides effective treatment of a disease or disorder in a patient. A therapeutically effective dose may vary from compound to compound, and from patient to patient, and may depend upon factors such as the condition of the patient and the route of delivery. A therapeutically effective dose may be determined in accordance with routine pharmacological procedures known to those skilled in the art.

### DETAILED DESCRIPTION

Reference is now made in detail to certain embodiments of the methods for treating patients by administering monomethyl fumarate. The disclosed embodiments are not intended to be limiting of the claims. To the contrary, the claims are intended to cover all alternatives, modifications, and equivalents.

### MMF-Glutathione Adducts

The monomethyl fumarate-glutathione adducts described herein are formed in vivo within a patient's body after administration of MMF to the patient according to the methods described herein. These MMF-glutathione adducts are one of two compounds, the compounds having the same molecular weight (437.43 daltons) but being regioisomers of one another, and diastereomers of either of the regioisomers. The two regioisomers differ from one another in the point of covalent attachment of the glutathione to the carbon backbone of the monomethyl fumarate. Thus, the first regioisomer (compound (1)) has the sulfur atom of glutathione attached to the 2-carbon of monomethyl fumarate whereas the second regioisomer (compound (2)) has the sulfur atom of glutathione attached to the 3-carbon of monomethyl fumarate. The two regioisomers have the following structures and chemical names:

[chemical structure]

4-(N-{(1R)-2-[1-carboxy-2-(methoxycarbonyl)ethylthio]-1-[N-(carboxymethyl)carbamoyl]ethyl}carbamoyl)(2S)-2-aminobutanoic acid (compound (1)); and

[chemical structure]

4-(N-{(1R)-2-[1-carboxy-2-(methoxycarbonyl)ethylthio]-1-[N-(carboxymethyl)carbamoyl]ethyl}carbamoyl)(2S)-2-aminobutanoic acid (compound (2)).

Each of compounds (1) and (2) has two disasteromers. Thus, compound (1) has two diastereomers which have the following structures and chemical names:

[chemical structure]

4-(N-{(1R)-2-[(1S)-1-carboxy-2-(methoxycarbonyl)ethylthio]-1-[N-(carboxymethyl)carbamoyl]ethyl}carbamoyl)(2S)-2-aminobutanoic acid (compound (1a)); and

[chemical structure]

4-(N-{(1R)-2-[(1R)-1-carboxy-2-(methoxycarbonyl)ethylthio]-1-[N-(carboxymethyl)carbamoyl]ethyl}carbamoyl)(2S)-2-aminobutanoic acid (compound (1b)).

Similarly, compound (2) has two diastereomers which have the following structures and chemical names:

[chemical structure]

4-(N-{(1R)-2-[(1S)-2-carboxy-1-(methoxycarbonyl)ethylthio]-1-[N-(carboxymethyl)carbamoyl]ethyl}carbamoyl)(2S)-2-aminobutanoic acid (compound (2a)); and

[chemical structure]

4-(N-{(1R)-2-[(1R)-2-carboxy-1-(methoxycarbonyl)ethylthio]-1-[N-(carboxymethyl)carbamoyl]ethyl}carbamoyl)(2S)-2-aminobutanoic acid (compound (2b)).

As described herein, the monomethyl fumarate-glutathione adducts may be in non-ionic forms, ionic forms, zwitterionic forms or salt forms.

### Methods

In accordance with a first aspect of the presently disclosed treatment methods, the MMF is administered in therapeutic amounts to treat a disease in a patient in need of such treatment. In some embodiments, the MMF is administered systemically. In some embodiments, the MMF is administered orally.

Specifically, the methods comprise administering MMF to a patient, at a dose and dosing frequency that achieves (i) a total area under a curve plotting average molar concentration of monomethyl fumarate-glutathione adducts in the blood plasma of the patient versus time (AUC_{molar-MMF-GA}); and (ii) a total area under a curve plotting average molar concentration of monomethyl fumarate in the blood plasma of the patient versus time (AUC_{molar-MMF}); wherein the ratio of AUC_{molar-MMF-GA}:AUC_{molar-MMF} is greater than 2%. In some embodiments, the ratio of AUC_{molar-MMF-GA}:AUC_{molar-MMF} is greater than 4%. In some embodiments, the ratio is from 4% to 100%. In some embodiments, the ratio is from 5% to 50%. In some embodiments, the ratio is from 5% to 20%. In some embodiments, the ratio is from 20% to 35%. In another embodiment, the ratio is from 35% to 50%.

In another aspect, the present disclosure discloses a method of administering a therapeutically effective amount of monomethyl fumarate to treat a disease in each patient of a population of patients in need of such treatment, comprising administering the monomethyl fumarate to each patient to achieve (i) a mean total area under a curve plotting average molar concentration of monomethyl fumarate-glutathione adducts in blood plasma of the patients versus time (mean AUC_{molar-MMF-GA}); and (ii) a mean total area under a curve plotting average molar concentration of monomethyl fumarate in the blood plasma of the patients versus time (mean AUC_{molar-MMF}); wherein a ratio of mean AUC_{molar-MMF-GA}:mean AUC_{molar-MMF} is greater than 2%.

In some embodiments, the ratio of AUC_{molar-MMF-GA}:AUC_{molar-MMF} is greater than 4%. In some embodiments, the ratio is from 4% to 100%. In some embodiments, the ratio is from 5% to 50%. In some embodiments, the ratio is from 5% to 20%. In some embodiments, the ratio is from 20% to 35%. In another embodiment, the ratio is from 35% to 50%.

In another aspect, the present disclosure provides methods of administering a therapeutically effective amount of monomethyl fumarate to treat a disease in a patient in need of such treatment, comprising administering the monomethyl fumarate to the patient at a monomethyl fumarate dose and dosing frequency that achieves formation of MMF-GA (monomethylfumarate-glutathione adducts) in blood plasma. In some embodiments, the mean maximum concentration (C_{max-MMF-GA}) of MMF-GA is at least 2% of the mean maximum concentration (C_{max-MMF}) of MMF.

In some embodiments, the mean maximum concentration (C_{max-MMF-GA}) of MMF-GA is at least 4% of the mean maximum concentration (C_{max-MMF}) of MMF.

In some embodiments, the mean maximum concentration (C_{max-MMF-GA}) of MMF-GA is from 4% to 50% of the mean maximum concentration (C_{max-MMF}) of MMF.

In some embodiments, the mean maximum concentration (C_{max-MMF-GA}) of MMF-GA is from 5% to 50% of the mean maximum concentration (C_{max-MMF}) of MMF.

In some embodiments, the mean maximum concentration (C_{max-MMF-GA}) of MMF-GA is from 5% to 20% of the mean maximum concentration (C_{max-MMF}) of MMF.

In some embodiments, the mean maximum concentration (C_{max-MMF-GA}) of MMF-GA is from 20% to 35% of the mean maximum concentration (C_{max-MMF}) of MMF.

In some embodiments, the mean maximum concentration (C_{max-MMF-GA}) of MMF-GA is from 35% to 50% of the mean maximum concentration (C_{max-MMF}) of MMF.

In order to achieve the pharmacokinetic values described herein, in some embodiments MMF is administered in different combinations of MMF dose and dosage frequency. Such combinations are given in Examples of the present disclosure as described herein.

The oral dosing of the formulation prepared according to Example 2 to fasted and fed healthy adult patients achieves the mean AUC_{molar-MMF-GA}:mean AUC_{molar-MMF} ratio of about 11% and about 15%, respectively (FIGS. 1 and 2).

The oral dosing of the formulation prepared according to Example 4 to fasted and fed healthy adult patients achieves the mean AUC_{molar-MMF-GA}:mean AUC_{molar-MMF} ratio of about 10% and about 17%, respectively (FIGS. 3 and 4).

The oral dosing of the formulation prepared according to Example 8 to fasted and fed healthy adult patients achieves the mean AUC_{molar-MMF-GA}:mean AUC_{molar-MMF} ratio of about 5% and about 12%, respectively (FIGS. 5 and 6).

The oral dosing of the formulation prepared according to Example 14 to fasted and fed healthy adult patients achieves the mean AUC_{molar-MMF-GA}:mean AUC_{molar-MMF} ratio of about 9% and about 40%, respectively (FIGS. 7 and 8).

### Pharmaceutical Compositions

Pharmaceutical compositions provided by the present disclosure may comprise a therapeutically effective amount of one or more active compounds together with a suitable amount of one or more pharmaceutically acceptable vehicles so as to provide a composition for proper administration to a patient. Suitable pharmaceutical vehicles are described in the art.

In certain embodiments, the active compound may be incorporated into pharmaceutical compositions to be administered orally. Oral administration of such pharmaceutical compositions may result in uptake of the active compound throughout the intestine and entry into the systemic circulation. Such oral compositions may be prepared in a manner known in the pharmaceutical art and comprise one or more active compounds and at least one pharmaceutically acceptable vehicle. Oral pharmaceutical compositions may include a therapeutically effective amount of one or more active compounds and a suitable amount of a pharmaceutically acceptable vehicle, so as to provide an appropriate form for administration to a patient.

The one or more active compounds may be incorporated into pharmaceutical compositions to be administered by any other appropriate route of systemic administration including intramuscular, intravenous and oral.

Pharmaceutical compositions comprising one or more therapeutically active compounds may be manufactured by means of conventional mixing, dissolving, granulating, dragee-making, levigating, emulsifying, encapsulating, entrapping, or lyophilizing processes. Pharmaceutical compositions may be formulated in a conventional manner using one or more physiologically acceptable carriers, diluents, excipients, or auxiliaries, which facilitate processing of the compound or crystalline forms thereof and one or more pharmaceutically acceptable vehicles into formulations that can be used pharmaceutically. Proper formulation is dependent upon the route of administration chosen. Pharmaceutical compositions provided by the present disclosure take the form of sustained-release formulations suitable for administration to a patient.

Pharmaceutical compositions provided by the present disclosure may be formulated in a unit dosage form. A unit dosage form refers to a physically discrete unit suitable as a unitary dose for patients undergoing treatment, with each unit containing a predetermined quantity of the active compound calculated to produce an intended therapeutic effect. A unit dosage form may be for a single daily dose, for administration 2 times per day, or one of multiple daily doses, e.g., 3 or more times per day. When multiple daily doses are used, a unit dosage form may be the same or different for each dose. One or more dosage forms may comprise a dose, which may be administered to a patient at a single point in time or during a time interval.

In certain embodiments, an oral dosage form provided by the present disclosure may be a controlled release dosage form. Controlled delivery technologies can improve the absorption of a drug in a particular region or regions of the gastrointestinal tract. Controlled drug delivery systems may be designed to deliver a drug in such a way that the drug level is maintained within a therapeutically effective window and effective and safe blood levels are maintained for a period as long as the system continues to deliver the drug with a particular release profile in the gastrointestinal tract. Controlled drug delivery may produce substantially constant blood levels of a drug over a period of time as compared to fluctuations observed with immediate release dosage forms. For some applications, maintaining a constant blood and tissue concentration throughout the course of therapy is the most desirable mode of treatment. Immediate release of drug may cause blood levels to peak above the level required to elicit a desired response, which may waste the drug and may cause or exacerbate toxic side effects. Controlled drug delivery can result in optimum therapy, and not only can reduce the frequency of dosing, but may also reduce the severity of side effects. Examples of controlled release dosage forms include dissolution controlled systems, diffusion controlled systems, ion exchange resins, osmotically controlled systems, erodable matrix systems, pH independent formulations, gastric retention systems, and the like.

An appropriate oral dosage form for a particular pharmaceutical composition provided by the present disclosure may depend, at least in part, on the gastrointestinal absorption properties of the active compound and/or the stability of the active compound in the gastrointestinal tract, the pharmacokinetics of the active compound and the intended therapeutic profile. An appropriate controlled release oral dosage form may be selected for a particular compound. For example, gastric retention oral dosage forms may be appropriate for compounds absorbed primarily from the upper gastrointestinal tract, and sustained release oral dosage forms may be appropriate for compounds absorbed primarily from the lower gastrointestinal tract. Certain compounds are absorbed primarily from the small intestine. In general, compounds traverse the length of the small intestine in about 3 to 5 hours. For compounds that are not easily absorbed by the small intestine or that do not dissolve readily, the window for active agent absorption in the small intestine may be too short to provide a desired therapeutic effect.

In certain embodiments, pharmaceutical compositions provided by the present disclosure may be practiced with dosage forms adapted to provide sustained release of MMF upon oral administration. Sustained release oral dosage forms may be used to release drugs over a prolonged time period and are useful when it is desired that a drug or drug form be delivered to the lower gastrointestinal tract, including the colon. Sustained release oral dosage forms include any oral dosage form that maintains therapeutic concentrations of a drug in a biological fluid such as the plasma, blood, cerebrospinal fluid, or in a tissue or organ for a prolonged time period. Sustained release oral dosage forms include diffusion-controlled systems such as reservoir devices and matrix devices, dissolution-controlled systems, osmotic systems, and erosion-controlled systems. Sustained release oral dosage forms and methods of preparing the same are well known in the art.

In certain embodiments, pharmaceutical compositions provided by the present disclosure may include any systemic dosage form of MMF, which when administered to a patient, achieves a total area under a curve plotting average molar concentration of monomethyl fumarate-glutathione adducts in blood plasma of the patient versus time (AUC_{molar-MMF-GA}) greater than 2% of a total area under a curve plotting average molar concentration of monomethyl fumarate in the blood plasma of the patient versus time (AUC_{molar-MMF}). In some embodiments, the MMF administration dosing and dosing frequency achieves a total area under a curve plotting average molar concentration of monomethyl fumarate-glutathione adducts in blood plasma of the patient versus time (AUC_{molar-MMF-GA}) greater than 4% of a total area under a curve plotting average molar concentration of monomethyl fumarate in the blood plasma of the patient versus time (AUC_{molar-MMF}). In some embodiments, the MMF administration dosing and dosing frequency achieves a total area under a curve plotting average molar concentration of monomethyl fumarate-glutathione adducts in blood plasma of the patient versus time (AUC_{molar-MMF-GA}) of from 4% to 100% of a total area under a curve plotting average molar concentration of monomethyl fumarate in the blood plasma of the patient versus time (AUC_{molar-MMF}). In some embodiments, the MMF administration dosing and dosing frequency achieves a total area under a curve plotting average molar concentration of monomethyl fumarate-glutathione adducts in blood plasma of the patient versus time (AUC_{molar-MMF-GA}) of from 5% to 50% of a total area under a curve plotting average molar concentration of monomethyl fumarate in the blood plasma of the patient versus time (AUC_{molar-MMF}). In some embodiments, the MMF administration dosing and dosing frequency achieves a total area under a curve plotting average molar concentration of monomethyl fumarate-glutathione adducts in blood plasma of the patient versus time (AUC_{molar-MMF-GA}) of from 5% to 20% of a total area under a curve plotting average molar concentration of monomethyl fumarate in the blood plasma of the patient versus time (AUC_{molar-MMF}). In some embodiments, the MMF administration dosing and dosing frequency achieves a total area under a curve plotting average molar concentration of monomethyl fumarate-glutathione adducts in blood plasma of the patient versus time (AUC_{molar-MMF-GA}) of from 20% to 35% of a total area under a curve plotting average molar concentration of monomethyl fumarate in the blood plasma of the patient versus time (AUC_{molar-MMF}). In some embodiments, the MMF administration dosing and dosing frequency achieves a total area under a curve plotting average molar concentration of monomethyl fumarate-glutathione adducts in blood plasma of the patient versus time (AUC_{molar-MMF-GA}) of from 35% to 50% of a total area under a curve plotting average molar concentration of monomethyl fumarate in the blood plasma of the patient versus time (AUC_{molar-MMF}).

In certain embodiments, pharmaceutical compositions provided by the present disclosure may include any systemic dosage form of MMF, and wherein, when administered to a population of patients, achieves a mean total area under a curve plotting average molar concentration of monomethyl fumarate-glutathione adducts in blood plasma of the patients versus time (mean AUC_{molar-MMF-GA}) greater than 2% of a mean total area under a curve plotting average molar concentration of monomethyl fumarate in the blood plasma of the patients versus time (mean AUC_{molar-MMF}). In some embodiments, the MMF administration dosing and dosing frequency achieves a mean AUC_{molar-MMF-GA} in blood plasma of the patients versus time greater than 5% of a mean AUC_{molar-MMF} in the blood plasma of the patients versus time. In some embodiments, the MMF administration dosing and dosing frequency achieves a mean AUC_{molar-MMF-GA} in blood plasma of the patients versus time of from 5% to 25% of a mean AUC_{molar-MMF} of the patients versus time. In some embodiments, the MMF administration dosing and dosing frequency achieves a mean AUC_{molar-MMF-GA} in blood plasma of the patients versus time of from 25% to 45% of a mean AUC_{molar-MMF} in the blood plasma of the patients versus time. In some embodiments, the MMF administration dosing and dosing frequency achieves a mean AUC_{molar-MMF-GA} in blood plasma of the patients versus time of from 45% to 65% of a mean AUC_{molar-MMF} in the blood plasma of the patients versus time. In some embodiments, the MMF administration dosing and dosing frequency achieves a mean AUC_{molar-MMF-GA} in blood plasma of the patients versus time of from 65% to 85% of a mean AUC_{molar-MMF} in the blood plasma of the patients versus time.

In some embodiments, the mean AUC_{molar-MMF-GA} in blood plasma is about 2 to 5% of the mean AUC_{molar-MMF} in the blood plasma. In some embodiments, the mean AUC_{molar-MMF-GA} in blood plasma is about 5 to 10% of the mean AUC_{molar-MMF}. In some embodiments, the mean AUC_{molar-MMF-GA} in blood plasma is about 10 to 15% of the mean AUC_{molar-MMF}. In some embodiments, the mean AUC_{molar-MMF-GA} in blood plasma is about 15 to 20% of the mean AUC_{molar-MMF}. In some embodiments, the mean AUC_{molar-MMF-GA} in blood plasma is about 20 to 25% of the mean AUC_{molar-MMF}. In some embodiments, the mean AUC_{molar-MMF-GA} in blood plasma is about 25 to 30% of the mean AUC_{molar-MMF}. In some embodiments, the mean AUC_{molar-MMF-GA} in blood plasma is about 30 to 35% of the mean AUC_{molar-MMF}. In some embodiments, the mean AUC_{molar-MMF-GA} in blood plasma is about 35 to 40% of the mean AUC_{molar-MMF}. In some embodiments, the mean AUC_{molar-MMF-GA} in blood plasma is about 5 to 20% of the mean AUC_{molar-MMF}.

In certain embodiments, pharmaceutical compositions provided by the present disclosure may include any systemic dosage form of MMF, which when administered to a patient, achieves formation of MMF-GA (monomethylfumarate-glutathione adducts) in blood plasma. In some embodiments, the maximum concentration (C_{max-MMF-GA}) of MMF-GA is at least 2% of the maximum concentration (C_{max-MMF}) of MMF. In some embodiments, C_{max-MMF-GA} is greater than 4% of C_{max-MMF}. In some embodiments, C_{max-MMF-GA} is about 4 to 50% of C_{max-MMF}. In some embodiments, C_{max-MMF-GA} is about 5 to 50% of C_{max-MMF}. In yet another embodiment, C_{max-MMF-GA} is about 5 to 20% of C_{max-MMF}. In some embodiments, C_{max-MMF-GA} is about 20 to 35% of C_{max-MMF}. In some embodiments, C_{max-MMF-GA} is about 35 to 50% of C_{max-MMF}.

In some embodiments, C_{max-MMF-GA} is about 2 to 5% of C_{max-MMF}. In some embodiments, C_{max-MMF-GA} is about 5 to 7% of C_{max-MMF}. In some embodiments, C_{max-MMF-GA} is about 7 to 9% of C_{max-MMF}. In some embodiments, C_{max-MMF-GA} is about 9 to 11% of C_{max-MMF}. In some embodiments, C_{max-MMF-GA} is about 11 to 13% of C_{max-MMF}. In some embodiments, C_{max-MMF-GA} is about 13 to 15% of C_{max-MMF}. In some embodiments, C_{max-MMF-GA} is about 15 to 17% of C_{max-MMF}. In some embodiments, C_{max-MMF-GA} is about 17 to 20% of C_{max-MMF}.

In certain embodiments, pharmaceutical compositions provided by the present disclosure may include any enteric-coated sustained release oral dosage form for administering the MMF. In some embodiments, the enteric-coated oral dosage form is administered to a patient at a dosing frequency of three times per day. In some embodiments, the enteric-coated oral dosage form is administered to a patient at a dosing frequency of twice per day. In some embodiments, the enteric-coated oral dosage form is administered to a patient at a dosing frequency of once per day.

In certain embodiments, pharmaceutical compositions provided by the present disclosure may include any non enteric-coated sustained release oral dosage form for administering the MMF. In some embodiments, the non enteric-coated oral dosage form is administered to a patient at a dosing frequency of three times per day. In some embodiments, the non enteric-coated oral dosage form is administered to a patient at a dosing frequency of twice per day. In some embodiments, the non enteric-coated oral dosage form is administered to a patient at a dosing frequency of once per day.

In certain embodiments, pharmaceutical compositions provided by the present disclosure may include any capsule oral dosage form for administering the MMF. In some embodiments, the capsule oral dosage form is administered to a patient at a dosing frequency of three times per day. In some embodiments, the capsule oral dosage form is administered to a patient at a dosing frequency of twice per day. In some embodiments, the capsule oral dosage form is administered to a patient at a dosing frequency of once per day.

In certain embodiments, pharmaceutical compositions provided by the present disclosure may include any suitable dosage forms that achieve the above described in vitro release profiles. Such dosage forms may be any systemic dosage forms, including sustained release enteric-coated oral dosage form and sustained release enteric-coated or non-enteric-coated oral dosage form. Examples of suitable dosage forms are described herein. Those skilled in the formulation art can develop any number of acceptable dosage forms given the dosage forms described in the examples as a starting point.

An appropriate dose of MMF may be determined according to any one of several well-established protocols. For example, animal studies such as studies using mice, rats, dogs, and/or monkeys may be used to determine an appropriate dose of a pharmaceutical compound. Results from animal studies may be extrapolated to determine doses for use in other species, such as for example, humans.

### Uses

The methods and compositions disclosed herein can be used to treat patients suffering from diseases, disorders, conditions, and symptoms for which MMF and/or other fumaric acid esters are known to provide or are later found to provide therapeutic benefit. MMF can be used to treat a disease chosen from adrenal leukodystrophy, AGE-induced genome damage, Alexanders Disease, alopecia greata, Alper's Disease, Alzheimer's disease, amyotrophic lateral sclerosis, angina pectoris, arthritis, asthma, balo concentric sclerosis, Behcet's disease, bollus pemphigoid, Canavan disease, cardiac insufficiency including left ventricular insufficiency, central nervous system vasculitis, Charcott-Marie-Tooth Disease, childhood ataxia with central nervous system hypomyelination, chronic idiopathic peripheral neuropathy, chronic obstructive pulmonary disease, Crohn's disease, cutaneous lupus, dermatitis (contact, acute and chronic), diabetic retinopathy, graft versus host disease, granulomas, hepatitis C viral infection, herpes simplex viral infection, human immunodeficiency viral infection, Huntington's disease, irritable bowel disorder, ischemia, Krabbe Disease, lichen planus, macular degeneration, mitochondrial encephalomyopathy, monomelic amyotrophy, multiple sclerosis, myocardial infarction, neurodegeneration with brain iron accumulation, neuromyelitis optica, neurosarcoidosis, NF-κB mediated diseases, optic neuritis, pareneoplastic syndromes, Parkinson's disease, Pelizaeus-Merzbacher disease, pemphigus, primary lateral sclerosis, progressive supranuclear palsy, psoriasis, pyoderma gangrenosum, reperfusion injury, retinopathia pigmentosa, sarcoidosis, Schilders Disease, subacute necrotizing myelopathy, susac syndrome, transplantation rejection, transverse myelitis, a tumor, ulcerative colitis or Zellweger's syndrome.

Methods of treating a disease in a patient provided by the present disclosure comprise administering to a patient in need of such treatment a therapeutically effective amount of MMF. These methods and pharmaceutical compositions provide therapeutic or prophylactic plasma and/or blood concentrations of MMF following administration to a patient. MMF may be administered in an amount and using a dosing schedule as appropriate for treatment of a particular disease. Daily doses of MMF may range from about 0.01 mg/kg to about 50 mg/kg, from about 0.1 mg/kg to about 50 mg/kg, from about 1 mg/kg to about 50 mg/kg, and in certain embodiments, from about 5 mg/kg to about 25 mg/kg. In certain embodiments, MMF may be administered at a dose over time from about 1 mg to about 5 g per day, from about 10 mg to about 4 g per day, in certain embodiments from about 20 mg to about 2 g per day, in certain embodiments from about 100 mg to about 1 g per day, in certain embodiments from about 200 mg to about 800 mg per day, in certain embodiments from about 300 mg to about 600 mg per day, and in certain embodiments from about 400 mg to about 500 mg per day. An appropriate dose of MMF may be determined based on several factors, including, for example, the body weight and/or condition of the patient being treated, the severity of the disease being treated, the incidence and/or severity of side effects, the manner of administration, and the judgment of the prescribing physician. Appropriate dose ranges may be determined by methods known to those skilled in the art.

MMF may be assayed in vitro and in vivo for the desired therapeutic or prophylactic activity prior to use in humans. In vivo assays, for example using appropriate animal models, may also be used to determine whether administration of MMF is therapeutically effective.

In certain embodiments, a therapeutically effective dose of MMF may provide therapeutic benefit without causing substantial toxicity including adverse side effects. Toxicity of MMF and/or metabolites thereof may be determined using standard pharmaceutical procedures and may be ascertained by those skilled in the art. The dose ratio between toxic and therapeutic effect is the therapeutic index. A dose of MMF may be within a range capable of establishing and maintaining a therapeutically effective circulating plasma and/or blood concentration of MMF that exhibits little or no toxicity.

MMF administration may be used to treat a disease chosen from adrenal leukodystrophy, AGE-induced genome damage, Alexanders Disease, alopecia greata, Alper's Disease, Alzheimer's disease, amyotrophic lateral sclerosis, angina pectoris, arthritis, asthma, balo concentric sclerosis, Behcet's disease, bollus pemphigoid, Canavan disease, cardiac insufficiency including left ventricular insufficiency, central nervous system vasculitis, Charcott-Marie-Tooth Disease, childhood ataxia with central nervous system hypomyelination, chronic idiopathic peripheral neuropathy, chronic obstructive pulmonary disease, Crohn's disease, cutaneous lupus, dermatitis (contact, acute and chronic), diabetic retinopathy, graft versus host disease, granulomas, hepatitis C viral infection, herpes simplex viral infection, human immunodeficiency viral infection, Huntington's disease, irritable bowel disorder, ischemia, Krabbe Disease, lichen planus, macular degeneration, mitochondrial encephalomyopathy, monomelic amyotrophy, multiple sclerosis, myocardial infarction, neurodegeneration with brain iron accumulation, neuromyelitis optica, neurosarcoidosis, NF-κB mediated diseases, optic neuritis, pareneoplastic syndromes, Parkinson's disease, Pelizaeus-Merzbacher disease, pemphigus, primary lateral sclerosis, progressive supranuclear palsy, psoriasis, pyoderma gangrenosum, reperfusion injury, retinopathia pigmentosa, sarcoidosis, Schilders Disease, subacute necrotizing myelopathy, susac syndrome, transplantation rejection, transverse myelitis, a tumor, ulcerative colitis or Zellweger's syndrome. The underlying etiology of any of the foregoing diseases being treated may have a multiplicity of origins. Further, in certain embodiments, a therapeutically effective amount of MMF may be administered to a patient, such as a human, as a preventative measure against the foregoing diseases and disorders. Thus, a therapeutically effective amount of MMF may be administered as a preventative measure to a patient having a predisposition for and/or history of adrenal leukodystrophy, AGE-induced genome damage, Alexanders Disease, alopecia greata, Alper's Disease, Alzheimer's disease, amyotrophic lateral sclerosis, angina pectoris, arthritis, asthma, balo concentric sclerosis, Behcet's disease, bollus pemphigoid, Canavan disease, cardiac insufficiency including left ventricular insufficiency, central nervous system vasculitis, Charcott-Marie-Tooth Disease, childhood ataxia with central nervous system hypomyelination, chronic idiopathic peripheral neuropathy, chronic obstructive pulmonary disease, Crohn's disease, cutaneous lupus, dermatitis (contact, acute and chronic), diabetic retinopathy, graft versus host disease, granulomas, hepatitis C viral infection, herpes simplex viral infection, human immunodeficiency viral infection, Huntington's disease, irritable bowel disorder, ischemia, Krabbe Disease, lichen planus, macular degeneration, mitochondrial encephalomyopathy, monomelic amyotrophy, multiple sclerosis, myocardial infarction, neurodegeneration with brain iron accumulation, neuromyelitis optica, neurosarcoidosis, NF-κB mediated diseases, optic neuritis, pareneoplastic syndromes, Parkinson's disease, Pelizaeus-Merzbacher disease, pemphigus, primary lateral sclerosis, progressive supranuclear palsy, psoriasis, pyoderma gangrenosum, reperfusion injury, retinopathia pigmentosa, sarcoidosis, Schilders Disease, subacute necrotizing myelopathy, susac syndrome, transplantation rejection, transverse myelitis, a tumor, ulcerative colitis or Zellweger's syndrome.

### Administration

MMF and pharmaceutical compositions thereof may be administered orally or by any other appropriate route suitable for systemic, as opposed to local, administration. For example, systemic administration can be by infusion or bolus injection, by absorption through epithelial or mucocutaneous linings (e.g., oral mucosa, rectal, and intestinal mucosa, etc.). Other suitable routes of systemic administration include, but are not limited to, intradermal, intramuscular, intraperitoneal, intravenous, subcutaneous, intranasal, epidural, oral, sublingual and inhalation.

The amount of MMF that will be effective in the treatment of a disease in a patient will depend, in part, on the nature of the condition and can be determined by standard clinical techniques known in the art. In addition, in vitro or in vivo assays may be employed to help identify optimal dosage ranges. A therapeutically effective amount of MMF to be administered may also depend on, among other factors, the subject being treated, the weight of the subject, the severity of the disease, the manner of administration, and the judgment of the prescribing physician. In the case of an MMF prodrug, for which MMF is the pharmacologically active metabolite, the amount of prodrug to be administered is generally determined by calculating the weight of any pharmacologically inactive promoiety that is cleaved during metabolism of the prodrug and then administering a MMF equivalent amount of the prodrug. For example, administering 250 mg of DMF is equivalent to administering 226 mg of MMF.

For systemic administration, a therapeutically effective dose may be estimated initially from in vitro assays. For example, a dose may be formulated in animal models to achieve a beneficial circulating composition concentration range. Initial doses may also be estimated from in vivo data, e.g., animal models, using techniques that are known in the art. Such information may be used to more accurately determine useful doses in humans. One having ordinary skill in the art may optimize administration to humans based on animal data.

A dose may be administered in a single dosage form or in multiple dosage forms. When multiple dosage forms are used the amount of compound contained within each dosage form may be the same or different. The amount of active compound contained in a dose may depend on the route of administration and whether the disease in a patient is effectively treated by acute, chronic, or a combination of acute and chronic administration.

In certain embodiments an administered dose is less than a toxic dose. Toxicity of the compositions described herein may be determined by standard pharmaceutical procedures in cell cultures or experimental animals, e.g., by determining the LD₅₀ (the dose lethal to 50% of the population) or the LD₁₀₀ (the dose lethal to 100% of the population). The dose ratio between toxic and therapeutic effect is the therapeutic index. In certain embodiments, MMF may exhibit a high therapeutic index. The data obtained from these cell culture assays and animal studies may be used in formulating a dosage range that is not toxic for use in humans. A dose of MMF provided by the present disclosure may be within a range of circulating concentrations in for example the blood, plasma, or central nervous system, that include the effective dose and that exhibits little or no toxicity. A dose may vary within this range depending upon the dosage form employed and the route of administration utilized. In certain embodiments, an escalating dose may be administered.

### Combination Therapy

Methods provided by the present disclosure further comprise administering one or more pharmaceutically active compounds in addition to MMF. Such compounds may be provided to treat the same disease or a different disease than the disease being treated with the MMF.

In certain embodiments, MMF may be used in combination with at least one other therapeutic agent. In certain embodiments, MMF may be administered to a patient together with another compound for treating diseases and conditions including: adrenal leukodystrophy, AGE-induced genome damage, Alexanders Disease, alopecia greata, Alper's Disease, Alzheimer's disease, amyotrophic lateral sclerosis, angina pectoris, arthritis, asthma, balo concentric sclerosis, Behcet's disease, bollus pemphigoid, Canavan disease, cardiac insufficiency including left ventricular insufficiency, central nervous system vasculitis, Charcott-Marie-Tooth Disease, childhood ataxia with central nervous system hypomyelination, chronic idiopathic peripheral neuropathy, chronic obstructive pulmonary disease, Crohn's disease, cutaneous lupus, dermatitis (contact, acute and chronic), diabetic retinopathy, graft versus host disease, granulomas, hepatitis C viral infection, herpes simplex viral infection, human immunodeficiency viral infection, Huntington's disease, irritable bowel disorder, ischemia, Krabbe Disease, lichen planus, macular degeneration, mitochondrial encephalomyopathy, monomelic amyotrophy, multiple sclerosis, myocardial infarction, neurodegeneration with brain iron accumulation, neuromyelitis optica, neurosarcoidosis, NF-κB mediated diseases, optic neuritis, pareneoplastic syndromes, Parkinson's disease, Pelizaeus-Merzbacher disease, pemphigus, primary lateral sclerosis, progressive supranuclear palsy, psoriasis, pyoderma gangrenosum, reperfusion injury, retinopathia pigmentosa, sarcoidosis, Schilders Disease, subacute necrotizing myelopathy, susac syndrome, transplantation rejection, transverse myelitis, a tumor, ulcerative colitis or Zellweger's syndrome.

MMF and the at least one other therapeutic agent may act additively or, and in certain embodiments, synergistically. The at least one additional therapeutic agent may be included in the same dosage form as MMF or may be provided in a separate dosage form. Methods provided by the present disclosure can further include, in addition to administering MMF, administering one or more therapeutic agents effective for treating the same or different disease than the disease being treated by MMF. Methods provided by the present disclosure include administration of MMF and one or more other therapeutic agents provided that the combined administration does not inhibit the therapeutic efficacy of the MMF and/or does not typically produce significant and/or substantial adverse combination effects.

In certain embodiments, dosage forms comprising MMF may be administered concurrently with the administration of another therapeutic agent, which may be part of the same dosage form as, or in a different dosage form than that comprising MMF. MMF may be administered prior or subsequent to administration of another therapeutic agent. In certain embodiments of combination therapy, the combination therapy may comprise alternating between administering MMF and a composition comprising another therapeutic agent, e.g., to minimize adverse drug effects associated with a particular drug. When MMF is administered concurrently with another therapeutic agent that potentially may produce an adverse drug effect including, but not limited to, toxicity, the other therapeutic agent may advantageously be administered at a dose that falls below the threshold at which the adverse drug reaction is elicited.

In certain embodiments, dosage forms comprising MMF may be administered with one or more substances to enhance, modulate and/or control release, bioavailability, therapeutic efficacy, therapeutic potency, stability, and the like of MMF. For example, to enhance the therapeutic efficacy of a MMF, the MMF may be co-administered with or a dosage form comprising MMF and one or more active agents to increase the absorption or diffusion of MMF from the gastrointestinal tract to the systemic circulation, or to inhibit degradation of the MMF in the blood of a patient. In certain embodiments, MMF may be co-administered with an active agent having pharmacological effects that enhance the therapeutic efficacy of MMF.

### EXAMPLES

The following examples illustrate various aspects of the disclosure. It will be apparent to those skilled in the art that many modifications, both to materials and methods, may be practiced without departing from the scope of the disclosure.

### Example 1

### Preparation of Sustained Release Dosage Form (Enteric Coated, 15% HPMC in Core, with Barrier Layer)

Delayed sustained release tablets containing the active compound were made having the ingredients shown in Table 1:

TABLE 1
Composition of Enteric Coated Sustained Release Tablet (15% HPMC in Core)

| | | | Quantity | Quantity |
|---|---|---|---|---|
| Component | Manufacturer | Role | (mg/tablet) | (% w/w) |
| Active Drug | XenoPort (Santa Clara, | MMF Source | 200.00 mg | 66.74 |
| | CA) | | 107 mg-eqs | |
| | | | MMF | |
| Hydroxypropyl | Ashland (Hopewell, | Binder | 6.19 | 2.06 |
| Cellulose | VA) | | | |
| Lactose | Foremost (Rothschild, | Filler | 44.95 | 15.00 |
| Monohydrate | WI) | | | |
| Hypromellose 2208 | Dow Chemical | Sustained | 44.95 | 15.00 |
| | (Midland, MI) | release agent | | |
| Silicon Dioxide | Cabot (Tuscola, IL) | Glidant | 0.60 | 0.20 |
| Magnesium Stearate | Mallinckrodt (St. | Lubricant | 3.00 | 1.00 |
| | Louis, MO) | | | |
| | | Total Core | 299.69 | 100.00 |
| Opadry 03O19184 | Colorcon (West Point, | Barrier coat | 7.13 | 2.38 |
| | PA) | | | |
| | | Total Barrier | 7.13 | 2.38 |
| | | Coating | | |
| Methacrylic Acid | Evonik Industries | Enteric | 24.20 | 8.08 |
| Co-polymer | (Essen, Germany) | polymer | | |
| Dispersion | | | | |
| Triethyl Citrate | Vertellus (Greensboro, | Plasticizer | 1.25 | 0.42 |
| | NC) | | | |
| PlasACRYL ™ T20 | Emerson Resources | Anti-tacking | 2.41 | 0.80 |
| | (Norristown, PA) | agent | | |
| | | Total Enteric | 27.87 | 9.30 |
| | | Coating | | |
| | | Total Tablet | 334.69 | 111.68 |

The tablets were made according to the following steps. The core tablets were prepared using a wet granulation process. The granulation was performed in two batches at 456 g per batch. The active drug and hydroxypropyl cellulose were passed through a conical mill with a 610 micron round holed screen. The active drug and hydroxypropyl cellulose were then combined in a Key KG-5 granulator bowl and mixed with water addition for approximately 7 minutes. The wet granules were dried in a Glatt GPCG-1 fluid bed dryer at 40° C. The two portions of dried granules were sized by passing through a conical mill with an approximately 1300 micron grater type screen. The milled granules were blended with the hypromellose 2208, silicon dioxide, and lactose monohydrate for 10 minutes in an 8 quart (7.6 l) V-blender. This blend was passed through an 850 micron mesh screen. The magnesium stearate was passed through a 600 micron mesh screen and blended with the additional core materials in the V-blender for 5 minutes. Core tablets (299.69 mg) were compressed using a GlobePharma Minipress II rotary tablet press with 8.6 mm round concave tooling. The core tablets had a final mean hardness of approximately 12 kp. An aqueous suspension was prepared by mixing with an impeller 63.8 g Opadry 03O19184 with 770.7 g of purified water. The water contained in the suspension is removed during the film coating process and therefore not included in the final formulation in Table 1. The tablets were coated with the aqueous suspension in an O' Hara Technologies Labcoat M coater with a 12″ (30.5 cm) diameter perforated pan until the desired weight gain of barrier coat was achieved. The coating process occurred at an inlet temperature of approximately 52° C. and an outlet temperature of 36° C. After coating, the tablets were dried for 2 hours at 40° C. An aqueous suspension was prepared by mixing with an impeller 405.1 g methacrylic acid copolymer dispersion, 6.3 g triethyl citrate, 60.6 g PlasACRYL™ T20 with 228.1 g water. The water contained in the methacrylic acid copolymer dispersion and the PlasACRYL™ T20 is removed during the film coating process and therefore not included in the final formulation in Table 1. The tablets were coated with the aqueous suspension in the O' Hara Technologies Labcoat M coater until the desired weight gain of enteric film was achieved. The coating process occurred at an inlet temperature of approximately 40° C. and an outlet temperature of 30° C. After coating, the tablets were dried for 2 hours at 40° C.

### Example 2

### Preparation of Delayed Sustained Release Dosage Form (Enteric Coated, 15% HPMC in Core, without Barrier Layer)

Delayed sustained release tablets containing the active drug were made having the ingredients shown in Table 2:

TABLE 2
Composition of Enteric Coated Sustained Release Tablet
(15% HPMC in Core, without Barrier Layer)

| | | | Quantity | Quantity |
|---|---|---|---|---|
| Component | Manufacturer | Role | (mg/tablet) | (% w/w) |
| Active Drug | XenoPort (Santa | MMF Source | 200.00 mg | 66.74 |
| | Clara, CA) | | 107 mg-eqs | |
| | | | MMF | |
| Hydroxypropyl | Ashland (Hopewell, | Binder | 6.18 | 2.06 |
| Cellulose | VA) | | | |
| Lactose Monohydrate | Foremost | Filler | 44.95 | 15.00 |
| | (Rothschild, WI) | | | |
| Hypromellose 2208 | Dow Chemical | Sustained | 44.95 | 15.00 |
| | (Midland, MI) | release agent | | |
| Silicon Dioxide | Cabot (Tuscola, IL) | Glidant | 0.60 | 0.20 |
| Magnesium Stearate | Mallinckrodt (St. | Lubricant | 3.00 | 1.00 |
| | Louis, MO) | | | |
| | | Total Core | 299.68 | 100.00 |
| Methacrylic Acid Co- | Evonik Industries | Enteric | 23.42 | 7.82 |
| polymer Dispersion | (Essen, Germany) | polymer | | |
| Triethyl Citrate | Vertellus | Plasticizer | 1.21 | 0.41 |
| | (Greensboro, NC) | | | |
| PlasACRYL ™ T20 | Emerson Resources | Anti-tacking | 2.33 | 0.78 |
| | (Norristown, PA) | agent | | |
| | | Total Coat | 27.90 | 9.00 |
| | | Total Tablet | 327.59 | 109.00 |

The tablets were made according to the following steps. The core tablets were prepared using a wet granulation process. The granulation was performed in two batches at 463.9 g per batch. The active drug and hydroxypropyl cellulose were passed through a conical mill with a 610 micron round holed screen. The active drug and hydroxypropyl cellulose were then combined in a Key KG-5 granulator bowl and mixed with water addition for approximately 10 minutes. The wet granules were dried in a Glatt GPCG-1 fluid bed dryer at 40° C. The two portions of dried granules were blended with silicon dioxide and sized by passing through a conical mill with an approximately 1300 micron grater type screen. The milled granules were blended with the hypromellose 2208 and lactose monohydrate for 10 minutes in an 8 quart (7.6 l) V-blender. This blend was passed through an 850 micron mesh screen. The magnesium stearate was passed through a 600 micron mesh screen and blended with the additional core materials in the V-blender for 5 minutes. Core tablets (299.68 mg) were compressed using a GlobePharma Minipress II rotary tablet press with 11/32″ round concave tooling. The core tablets had a final mean hardness of approximately 11 kp. An aqueous suspension was prepared by mixing with an impeller 578.7 g methacrylic acid copolymer dispersion, 9.0 g triethyl citrate, 86.5 g PlasACRYL™ T20 with 325.8 g water. The water contained in the methacrylic acid copolymer dispersion and the PlasACRYL™ T20 is removed during the film coating process and therefore not included in the final formulation in Table 2. The tablets were coated with the aqueous suspension in the O' Hara Technologies Labcoat M coater until the desired weight gain of enteric film was achieved. The coating process occurred at an inlet temperature of approximately 41° C. and an outlet temperature of 31° C. After coating, the tablets were dried for 2 hours at 40° C.

### Example 3

### Safety, Tolerability, and Pharmacokinetics of Example 2 Dosage Form

A randomized, double-blind crossover, food effect, single-dose study of the safety, tolerability, and pharmacokinetics of an oral dosage form of Example 2 in healthy adult subjects was conducted. Twenty-four healthy adult volunteers (males and females) participated in the study. Twelve of the subjects received a dosage form of Example 2, once in a fed condition and once in a fasted condition, with a two-week washout between treatments. The fasted dosing was achieved by dosing the subject following an overnight fast while the fed dosing was achieved by dosing the subject after consuming a high fat-content breakfast. The tested dosage forms contained 200 mg of the active drug, 107 mg equivalents of MMF.

Blood samples were collected from all subjects prior to dosing, and at 0.5, 1, 1.5, 2, 3, 4, 5, 6, 7, 8, 10, 12, 16, 24, 30, 36, 48, 60, 72, 84, 96, 108 and 120 hours after dosing. Urine samples were collected from all subjects prior to dosing, and complete urine output was obtained at the 0-4, 4-8, 8-12, 12-24, 24-36, 36-48, 48-72, 72-96 and 96-120 hour intervals after dosing. Blood samples were quenched immediately with acetonitrile and frozen. Sample aliquots were prepared for analysis of (i) MMF, (ii) the active drug, and (iii) other potential metabolites using sensitive and specific LC/MS/MS methods.

The plasma concentrations of MMF and MMF-glutathione adducts following oral dosing of the formulation prepared according to Example 2 to fasted and fed healthy adult patients is shown in FIG. 1 and FIG. 2, respectively. Table 3 shows the mean (SD) pharmacokinetic data in fed and fasted patients. The “Mean AUC_{molar-MMF}” and “Mean AUC_{molar-MMF-GA}” values presented in the table are the average of the individual values for these parameters in each subject. Similarly, the “Mean AUC_{molar-MMF-GA}:Mean AUC_{molar-MMF} Ratio (%)” presented in the table is the average of the individual ratio values calculated in each subject, and therefore is not identical to the ratio of the “Mean AUC_{molar-MMF}” and “Mean AUC_{molar-MMF-GA}” values.

TABLE 3
PK Data

| | | | | Mean |
|---|---|---|---|---|
| | | Mean | Mean | AUC_{molar-MMF-GA}:mean |
| | | AUC_{molar-MMF-GA} | AUC_{molar-MMF} | AUC_{molar-MMF} |
| N | Food | (μM · hr) | (μM · hr) | Ratio (%) |
| 12 | Fast- | 0.356 | 3.00 | 11.1 |
| | ed | | | |
| 12 | Fed | 0.398 | 2.45 | 15.3 |

The drug was well tolerated during the trial. All 12 subjects completed the dosing period. All adverse events were mild.

### Example 4

### Preparation of Dosage Form Comprising HPMC-Based Capsule Containing Enteric-Coated Pellets

Size 00 VCaps Plus capsules containing 477 mg of extended-release drug-containing pellets were manufactured with the formulation shown in Table 4:

TABLE 4
Composition of VCaps Plus Capsule

| | | | Quantity | Quantity |
|---|---|---|---|---|
| Component | Manufacturer | Role | (mg/tablet) | (% w/w) |
| Active Drug | Cambridge | MMF Source | 200.00 mg | 60.00 |
| | (Germantown, WI) | | 107 mg-eqs | |
| | | | MMF | |
| Microcrystalline Cellulose | FMC | Filler | 133.33 | 40.00 |
| | (Newark, DE) | | | |
| | | Total Pellet | 333.33 | 100.00 |
| | | Core | | |
| Ethylcellulose | Ashland | Water-insoluble | 20.56 | 6.17 |
| | (Hopewell VA) | coating agent | | |
| Hydroxypropyl Cellulose | Ashland | Water soluble | 5.00 | 1.50 |
| | (Hopewell VA) | coating agent | | |
| Talc | Luzenac | Anti-tacking | 5.00 | 1.50 |
| | (Houston TX) | agent | | |
| Dibutyl sebacate | Vertellus | Plasticizer | 2.78 | 0.83 |
| | (Greensboro, NC) | | | |
| | | Total Barrier/ | 33.33 | 10.00 |
| | | Sustained | | |
| | | Release | | |
| | | Coating | | |
| Methacrylic Acid Co- | Evonik | Enteric coating | 88.55 | 24.15 |
| polymer Dispersion | (Darmstadt, | agent | | |
| | Germany) | | | |
| Triethyl Citrate | Vertellus | Plasticizer | 14.30 | 3.90 |
| | (Greensboro, NC) | | | |
| PlasACRYL T20 | Emerson | Anti-tacking | 7.15 | 1.95 |
| | (Norristown, PA) | agent | | |
| | | Total Enteric | 110.00 | 30.00 |
| | | Coating | | |
| VCaps Plus Size 00 | Capsugel | Capsule | 111-125 | 23.29-26.22 |
| Capsule | (Puebla, Mexico) | | | |

The capsules were manufactured according to the following process. An extrusion/spheronization process was selected for the manufacture of the core pellets for the capsules. The drug was first screened then mixed with microcrystalline cellulose. This blend was then formed into a wet mass with the addition of aqueous acetate buffer (pH 3.5) and the mass then extruded through a 1.0 mm screen and the extrudates were spheronized (at 1200 rpm for 3 minutes) to form the core pellets. These core pellets are then classed to retain the pellets within 0.85 mm to 1.4 mm before the next processing step. The pellets were then coated with the target amount of the sustained release membrane using a hydroalcoholic mixture of ethylcellulose and hydroxypropyl cellulose. This coating was performed in a Wurster-type coater (product temperature at 30° C. and spray rate at 10 g/minute). The overall coating time was approximately 2 hours. The coated pellets were dried further in an oven to remove any residual solvent. The dried sustained release film-coated pellets were then enteric coated to the target amount by aqueous film coating in a Wurster-type coater (product temperature at 30° C. and a spray rate at 10 g/min). The overall coating time was approximately 2 hours. The capsules were then filled with the appropriate amount of pellets to achieve the desired dose strength.

### Example 5

### Safety, Tolerability, and Pharmacokinetics of Example 4

### Enteric-Coated Pellets in a Capsule Dosage Form

A randomized, double-blind crossover, food effect, single-dose study of the safety, tolerability, and pharmacokinetics of a sustained release oral dosage form of Example 4 in healthy adult subjects was conducted. Twelve healthy adult volunteers (males and females) participated in the study. All twelve subjects received a dosage form of Example 4, once in a fed condition and once in a fasted condition, with a two-week washout between treatments. The fasted dosing was achieved by dosing the subject following an overnight fast while the fed dosing was achieved by dosing the subject after consuming a high fat-content breakfast. The dosage form contained 200 mg of drug, 107 mg equivalents of MMF.

Blood samples were collected from all subjects prior to dosing, and at 0.5, 1, 1.5, 2, 3, 4, 5, 6, 7, 8, 10, 12, 16, 24, 30, 36, 48, 60, 72, 84, 96, 108 and 120 hours after dosing. Urine samples were collected from all subjects prior to dosing, and complete urine output was obtained at the 0-4, 4-8, 8-12, 12-24, 24-36, 36-48, 48-72, 72-96 and 96-120 hour intervals after dosing. Blood samples were quenched immediately with acetonitrile and frozen. Sample aliquots were prepared for analysis of (i) MMF, (ii) drug, and (iii) other potential metaboliltes using sensitive and specific LC/MS/MS methods.

The plasma molar concentrations of MMF and MMF-glutathione adducts following oral dosing of the formulation prepared according to Example 4 to fasted and fed healthy adult patients is shown in FIG. 3 and FIG. 4, respectively. Table 5 shows the mean (SD) pharmacokinetic data for the Example 4 dosage forms in fed and fasted patients. The “Mean AUC_{molar-MMF}” and “Mean AUC_{molar-MMF-GA}” values presented in the table are the average of the individual values for these parameters in each subject. Similarly, the “Mean AUC_{molar-MMF-GA}: Mean AUC_{molar-MMF} Ratio (%)” presented in the table is the average of the individual ratio values calculated in each subject, and therefore is not identical to the ratio of the “Mean AUC_{molar-MMF}” and “Mean AUC_{molar-MMF-GA}” values.

TABLE 5
PK Data for Capsule Dosage Form

| | | | | Mean |
|---|---|---|---|---|
| | | Mean | Mean | AUC_{molar-MMF-GA}:mean |
| | | AUC_{molar-MMF-GA} | AUC_{molar-MMF} | AUC_{molar-MMF} |
| N | Food | (μM · hr) | (μM · hr) | Ratio (%) |
| 12 | Fast- | 0.131 | 1.72 | 9.82 |
| | ed | | | |
| 12 | Fed | 0.481 | 3.00 | 16.9 |

The drug was well tolerated during the trial. All 12 subjects completed the dosing period. All adverse events were mild.

### Example 6

### Preparation of Compression Coated Tablet Dosage Form (Non-Enteric Coated, 8% HPMC in Core)

Compression coated tablets containing drug were made having the ingredients shown in Table 6:

TABLE 6
Composition of Compression Coated Tablet Dosage Form
(Non-Enteric Coated, 8% HPMC in Core)

| | | | Quantity | |
|---|---|---|---|---|
| | | | (mg/ | Quantity |
| Component | Manufacturer | Role | tablet) | (% w/w) |
| Active Drug | XenoPort (Santa | MMF | 100.00 mg | 29.19 |
| | Clara, CA) | Source | 53.5 mg- | |
| | | | eqs MMF | |
| Hydroxypropyl | Aqualon (Hopewell, | Binder | 3.12 | 0.91 |
| Cellulose | VA) | | | |
| Hypromellose | Dow Chemical | Sustained | 9.14 | 2.67 |
| 2208 | (Midland, MI) | Release | | |
| (100000 | | Polymer | | |
| mPa · s) | | | | |
| Silicon Dioxide | Cabot (Tuscola, IL) | Glidant | 0.23 | 0.06 |
| Magnesium | Mallinckrodt (St. | Lubricant | 1.71 | 0.50 |
| Stearate | Louis, MO) | | | |
| | | Total Core | 114.20 | 33.33 |
| Lactose | Foremost | Filler | 157.60 | 46.00 |
| Hydrate | (Rothschild, WI) | | | |
| Hypromellose | Dow Chemical | Sustained | 68.52 | 20.00 |
| 2208 | (Midland, MI) | Release | | |
| (100 mPa · s) | | Polymer | | |
| Magnesium | Mallinckrodt (St. | Lubricant | 2.28 | 0.67 |
| Stearate | Louis, MO) | | | |
| | | Total | 228.40 | 66.67 |
| | | Mantle | | |
| | | Total | 342.60 | 100.00 |
| | | Tablet | | |

The tablets were made according to the following steps. The core tablets were prepared using a wet granulation process. The granulation batch size was 680 g. Drug was passed through the Quadro Comil U5 with an 813 micron screen at 2000 rpm. Hydroxypropyl cellulose was passed through a 600 micron mesh screen. Drug and hydroxypropyl cellulose were granulated with purified water using a Diosna P1/6 equipped with a 4 L bowl. The wet granules were screened through an 1180 micron mesh screen and dried on trays in an oven at 30° C. for 6 hours.

The core blend batch size was 5 g. The dried granules, hydroxypropylmethyl cellulose (i.e., hypromellose 2208 having 100000 mPa·s viscosity), and the silicon dioxide were then passed through a 600 micron mesh screen, combined in a glass jar and blended on a Turbula mixer for 5 minutes. Magnesium stearate was passed through a 250 micron screen and added to the blend before blending an additional 1.5 minutes. Core tablets (114.2 mg) were compressed using a Carver Press with ¼ inch (6.35 mm) round standard concave tooling at 0.4 metric ton (MT) force. The core tablets had a final hardness of approximately 7.6 kp (˜74 Newtons).

The mantle blend was prepared using a direct compression process and a batch size of 10 g. The hypromellose 2208 (100 MPa·s viscosity) and lactose hydrate were passed through a 600 micron mesh screen, combined in a glass jar and blended on a Turbula mixer for 5 minutes. Magnesium stearate was passed through a 250 micron screen and added to the blend and blended an additional 1.5 minutes. The mantle blend was then applied to the core tablets using the Carver Press with ⅜ inch (9.53 mm) round standard concave tooling. Half the mantle blend (114.2 mg) was weighed out, added to the die, and tamped slightly to flatten. Then, the core tablet was placed into the die and pressed down gently into the mantle blend. The second half of the mantle blend (114.2 mg) was then added on top of the core tablet and the mantle was compressed using 1.5 MT force. The final compression coated tablets had a total weight of 342.6 mg with a drug loading of 100 mg (29.19%) or 53.5 mg equivalents of MMF. The tablets had a final hardness around 14.7 kp (˜144 Newtons).

### Example 7

### Preparation of Compression Coated Tablet Dosage Form (Non-Enteric Coated, 30% HPMC in Mantle)

Compression coated tablets containing drug were made having the ingredients shown in Table 7:

TABLE 7
Composition of Compression Coated Tablet Dosage Form (Non-Enteric Coated,
30% HPMC in Mantle)

| | | | Quantity | Quantity |
|---|---|---|---|---|
| Component | Manufacturer | Role | (mg/tablet) | (% w/w) |
| Active Drug | XenoPort (Santa Clara, | MMF Source | 100.00 | 31.78 |
| | CA) | | | |
| Hydroxypropyl | Aqualon (Hopewell, | Binder | 3.12 | 0.99 |
| Cellulose | VA) | | | |
| Silicon Dioxide | Cabot (Tuscola, IL) | Glidant | 0.21 | 0.06 |
| Magnesium Stearate | Mallinckrodt (St. | Lubricant | 1.57 | 0.50 |
| | Louis, MO) | | | |
| | | Total Core | 104.90 | 33.33 |
| Lactose Hydrate | Foremost | Filler | 144.76 | 46.00 |
| | (Rothschild, WI) | | | |
| Hypromellose 2208 | Dow Chemical | Sustained | 62.94 | 20.00 |
| (100000 mPa · s) | (Midland, MI) | Release | | |
| | | Polymer | | |
| Magnesium Stearate | Mallinckrodt (St. | Lubricant | 2.10 | 0.67 |
| | Louis, MO) | | | |
| | | Total Mantle | 209.80 | 66.67 |
| | | Total Tablet | 314.70 | 100.00 |

The tablets were made according to the following steps. The core tablets were prepared using a wet granulation process. The granulation batch size was 680 g. Drug was passed through the Quadro Comil U5 with an 813 micron screen at 2000 rpm. Hydroxypropyl cellulose was passed through a 600 micron mesh screen. Drug and hydroxypropyl cellulose were granulated with purified water using a Diosna P1/6 equipped with a 4 L bowl. The wet granules were screened through an 1180 micron mesh screen and dried on trays in an oven at 30° C. for 6 hours.

The core blend batch size was 5 g. The dried granules and the silicon dioxide were then passed through a 600 micron mesh screen, combined in a glass jar and blended on a Turbula mixer for 5 minutes. Magnesium stearate was passed through a 250 micron screen and added to the blend before blending an additional 1.5 minutes. Core tablets (104.9 mg) were compressed using a Carver Press with ¼ inch (6.35 mm) round standard concave tooling at 0.4 metric ton (MT) force. The core tablets had a final hardness of approximately 6.1 kp (˜60 Newtons).

The mantle blend was prepared using a direct compression process and a batch size of 100 g. The hydroxypropylmethyl cellulose (i.e., hypromellose 2208 having 100000 MPa·s viscosity) and lactose hydrate were passed through a 600 micron mesh screen, combined in a 1 quart (0.95 l) V-blender and blended for 10 minutes. Magnesium stearate was passed through a 250 micron screen and added to the blend and blended an additional 4 minutes. The mantle blend was then applied to the core tablets using the Carver Press with ⅜ inch (9.53 mm) round standard concave tooling. Half the mantle blend (104.9 mg) was weighed out, added to the die, and tamped slightly to flatten. Then, the core tablet was placed into the die and pressed down gently into the mantle blend. The second half of the mantle blend (104.9 mg) was then added on top of the core tablet, and the mantle was compressed using 1.5 MT force. The final compression coated tablets had a total weight of 314.7 mg with a drug loading of 100 mg (31.78%) or 53.5 mg equivalents of MMF. The tablets had a final hardness around 13.1 kp (˜128 Newtons).

### Example 8

### Composition of Compression Coated Tablet Dosage Form (Non-Enteric Coated, 8% HPMC in Core)

Compression coated tablets were made having the ingredients shown in Table 8:

TABLE 8
Composition of Compression Coated Tablet Dosage Form (Non-Enteric Coated,
8% HPMC in Core)

| | | | Quantity | Quantity |
|---|---|---|---|---|
| Component | Manufacturer | Role | (mg/tablet) | (% w/w) |
| Active Drug | Cambridge Major | MMF Source | 100.0 | 27.59 |
| | (Germantown, WI) | | | |
| Hydroxypropyl | Aqualon | Binder | 3.1 | 0.86 |
| Cellulose | (Hopewell, VA) | | | |
| Hypromellose 2208 | Dow Chemical | Sustained | 9.1 | 2.51 |
| (100000 mPa · s) | (Midland, MI) | Release Polymer | | |
| Silicon Dioxide | Evonik | Glidant | 0.6 | 0.17 |
| | (Rheinfelden, | | | |
| | Germany) | | | |
| Magnesium Stearate | Mallinckrodt (St. | Lubricant | 1.7 | 0.47 |
| | Louis, MO) | | | |
| | | Total Core | 114.5 | 31.59 |
| Lactose Hydrate | Foremost | Filler | 164.8 | 45.47 |
| | (Rothschild, WI) | | | |
| Hypromellose 2208 | Dow Chemical | Sustained | 80.6 | 22.24 |
| (100 mPa · s) | (Midland, MI) | Release Polymer | | |
| Magnesium Stearate | Mallinckrodt (St. | Lubricant | 2.5 | 0.69 |
| | Louis, MO) | | | |
| | | Total Mantle | 247.9 | 68.41 |
| | | Total Tablet | 362.4 | 100.00 |

The tablets were made according to the following steps. The core tablets were prepared using a wet granulation process. The granulation was performed in 2 batches at 494.88 g each. Drug was passed through a 1.0 mm mesh screen. Hydroxypropyl cellulose was passed through a 600 micron mesh screen. Drug and hydroxypropyl cellulose were combined in a 3 L bowl and mixed for 10 minutes using the Quintech granulator. The mixture was then transferred to a 2 L bowl granulated with purified water using the Quintech granulator. The wet granules were screened through a 2000 micron mesh screen and dried on trays in an oven at 30° C. for 4 hours 20 minutes. The dried granules were then passed through an 850 micron screen.

The core blend batch size was 1099.2 g. The hydroxypropylmethyl-cellulose (i.e., Hypromellose 2208 having 100000 mPa·s viscosity) and the silicon dioxide were combined, passed through a 600 micron mesh screen, and added to the dry granules in a 5 L cube blender and blended for 10 minutes at 25 rpm. Magnesium stearate was passed through a 600 micron screen and added to the blend before blending an additional 4 minutes at 25 rpm. Core tablets (114.5 mg) were compressed using a Manesty F3 tablet press with 6.0 mm round concave tooling. The core tablets had a final mean hardness between 8.1 to 10.2 kp (79-100 Newtons).

The mantle blend was prepared using a direct compression process and a batch size of 5.0 kg. The hypromellose 2208 (100 MPa·s viscosity) and lactose hydrate were combined and passed through a 600 micron mesh screen, placed in and blended on the Tumblemix 18 L Bin Blender for 8.5 minutes at 30 rpm. Magnesium stearate was passed through a 600 micron screen and added to the blend and blended an additional 3.5 minutes. The mantle blend was then applied to the core tablets using a Kikusui tablet press (Kikusui Seisakusho Ltd., Kyoto, Japan) specially designed for the manufacture of compression coated tablets. Compression was completed using 9.5 mm round concave tooling and approximately 1000 kp force. The final compression coated tablets had a total weight of 362.4 mg with a drug loading of 100 mg (27.59%) or 53.5 mg equivalents of MMF. The compression coated tablets had a final mean hardness between 10.9 to 14.0 kp (107-137 Newtons).

### Example 9

### Preparation of Compression Coated Tablet Dosage Form (Non-Enteric Coated, 10% HPMC in Core)

Two different tablet formulations were made according to the procedure outlined in Example 6, but with differing levels of hypromellose 2208 (100000 MPa·s viscosity) in the core, i.e., compared to the Example 6 tablets. Thus, the Example 6 tablets contained 8 wt % HPMC in the core while the Example 9 tablets contained 10 wt % HPMC in the core, respectively. The tablet formulations, including the Example 6 tablet formulation for reference, are shown in Table 9.

TABLE 9
Composition of Compression Coated Tablet Dosage Forms
(Non-Enteric Coated, 8% and 10% HPMC in Core)

| | Quantity | Quantity | Quantity | Quantity |
|---|---|---|---|---|
| | (mg/tablet) | (% w/w) | (mg/tablet) | (% w/w) |
| Component | Example 6 | | Example 9 | |
| Active Drug/MMF | 100.00 mg | 29.19 | 100.00 mg | 28.55 |
| Source | 53.5 mg-eqs | | 53.5 mg-eqs | |
| | MMF | | MMF | |
| Hydroxypropyl | 3.12 | 0.91 | 3.10 | 0.88 |
| Cellulose | | | | |
| Hypromellose 2208 | 9.14 | 2.67 | 11.67 | 3.33 |
| (100000 mPa · s) | | | | |
| Silicon Dioxide | 0.23 | 0.06 | 0.23 | 0.07 |
| Magnesium Stearate | 1.71 | 0.50 | 1.75 | 0.50 |
| Total Core | 114.20 | 33.33 | 116.75 | 33.33 |
| Lactose Hydrate | 157.60 | 46.00 | 161.12 | 46.00 |
| Hypromellose 2208 | 68.52 | 20.00 | 70.05 | 20.00 |
| (100 mPa · s) | | | | |
| Magnesium Stearate | 2.28 | 0.67 | 2.33 | 0.67 |
| Total Mantle | 228.40 | 66.67 | 233.50 | 66.67 |
| Total Tablet | 342.60 | 100.00 | 350.25 | 100.00 |

### Example 10

### Preparation of Sustained Release Compression Coated Tablet Dosage Forms (Non-Enteric Coated)

Tablets were made with hypromellose 2208 of different viscosities in the mantle: Example 10a (4000 mPa·s), and Example 10b (a combination of 100 mPa·s and 4000 mPa·s to give an effective viscosity of ˜2000 mPa·s). The formulation details are shown in Table 10.

TABLE 10
Composition of Sustained Release Tablet Dosage Forms
(Non-Enteric Coated)

| | Quantity | Quantity | Quantity | Quantity |
|---|---|---|---|---|
| | (mg/tablet) | (% w/w) | (mg/tablet) | (% w/w) |
| Component | Example 10a | | Example 10b | |
| Active Drug/MMF | 200.00 mg | 32.00 | 200.00 mg | 32.00 |
| Source | 107 mg-eqs | | 107 mg-eqs | |
| | MMF | | MMF | |
| Hydroxypropyl | 6.20 | 1.00 | 6.20 | 1.00 |
| Cellulose | | | | |
| Magnesium Stearate | 2.10 | 0.30 | 2.10 | 0.30 |
| Total Core | 208.30 | 33.30 | 208.30 | 33.30 |
| Lactose Hydrate | 308.30 | 49.30 | 308.30 | 49.30 |
| Hypromellose 2208 | 0.00 | 0.00 | 52.05 | 8.35 |
| (100 mPa · s) | | | | |
| Hypromellose 2208 | 104.10 | 16.70 | 52.05 | 8.35 |
| (4000 mPa · s) | | | | |
| Magnesium Stearate | 4.20 | 0.70 | 4.20 | 0.70 |
| Total Mantle | 416.60 | 66.70 | 416.60 | 66.70 |
| Total Tablet | 624.90 | 100.00 | 624.90 | 100.00 |

The tablets were made according to the following steps. The core tablets were prepared using a wet granulation process. The granulation batch size was 170 g. Drug was passed through the Quadro Comil U5 with an 813 micron screen at 2000 rpm. Hydroxypropyl cellulose was passed through a 500 micron mesh screen. Drug and hydroxypropyl cellulose were granulated with purified water using a Diosna P1/6 equipped with a 1 L bowl. The wet granules were screened through an 1180 micron mesh screen and dried on trays in an oven at 30° C. for 3 hours 48 minutes.

The core blend batch size was 20.0 g. The dried granules and magnesium stearate were combined in a glass bottle and blended on a Turbula mixer for 2 minutes. Core tablets (208.3 mg) were compressed using a Manesty FlexiTab single station tablet press with 5/16 inch (7.9 mm) round standard concave tooling at forces ranging from 9.9 to 14.0 kN. The core tablets had a final mean hardness of 8.4 kp (˜82 Newtons).

The mantle blend was prepared using a direct compression process and a batch size of either 10 g (Example 10b) or 20 g (Example 10a). The hypromellose 2208 and lactose hydrate were passed through a 600 micron mesh screen, combined in a glass bottle and blended on a Turbula mixer for either 10 (Example 10a), or 5 (Example 10b) minutes. In each case, magnesium stearate was passed through a 250 micron screen and added to the blend and blended an additional 1.5 minutes. The mantle blend was then applied to the core tablets using the Carver Press with 7/16 inch (11.1 mm) round standard concave tooling. Half the mantle blend (208.3 mg) was weighed out, added to the die, and tamped slightly to flatten. Then, the core tablet was placed into the die and pressed down gently into the mantle blend. The second half of the mantle blend (208.3 mg) was then added on top of the core tablet and the mantle was compressed using 2.0 metric ton (MT) force. The final compression coated tablets had a total weight of 624.9 mg with a drug loading of 200 mg (32.00%) or 107 mg equivalents of MMF. The tablets had a final hardness of about 18.3 to 19.5 kp (˜179 to 191 Newtons).

### Example 11

### Preparation of Sustained Release Compression Coated Tablet Dosage Forms (Non-Enteric Coated with 5 wt % Hypromellose 2208 (100000 mPa·s) in the Core and 40% Hypromellose 2208 (100 MPa·s) in the Mantle)

Tablets were made according to the procedure outlined in Example 6, but with 5 wt % hypromellose 2208 (100000 mPa·s) in the core and 40% of hypromellose 2208 (100 MPa·s) in the mantle: The tablet formulation is shown in Table 11.

TABLE 11
Composition of Sustained Release Compression Coated Tablet
Dosage Forms (Non-Enteric Coated with 5 wt % hypromellose 2208
(100000 mPa · s) in the core and 40% hypromellose 2208
(100 MPa · s) in the mantle)

| | |
|---|---|
| Quantity (mg/tablet) | Quantity (% w/w) |

| | |
|---|---|
| Component | Example 11 |

| | | |
|---|---|---|
| Active Drug/MMF | 100.00 mg | 30.17 |
| Source | 53.5 mg-eqs MMF | |
| Hydroxypropyl | 3.10 | 0.93 |
| Cellulose | | |
| Hypromellose 2208 | 5.52 | 1.66 |
| (100000 MPa · s) | | |
| Silicon Dioxide | 0.22 | 0.07 |
| Magnesium Stearate | 1.66 | 0.50 |
| Total Core | 110.50 | 33.33 |
| Lactose Hydrate | 130.39 | 39.33 |
| Hypromellose 2208 | 88.40 | 26.67 |
| (100 MPa · s) | | |
| Magnesium Stearate | 2.21 | 0.67 |
| Total Mantle | 221.00 | 66.67 |
| Total Tablet | 331.50 | 100.00 |

### Example 12

### Preparation of Sustained Release Tablet Dosage Forms (Non-Enteric Coated Formulation with No Hypromellose in the Core and Thin Mantle)

The mantle-to-core weight ratio was decreased from 2 to 1.5 in the tablet formulation shown in Table 12.

TABLE 12
Composition of SR Tablet Dosage Form (Non-Enteric Coated)

| | Quantity (mg/tablet) | Quantity (% w/w) |
|---|---|---|
| Component | Example 12 | |
| Active Drug/MMF | 100.00 mg | 38.37 |
| Source | 53.5 mg-eqs MMF | |
| Hydroxypropyl Cellulose | 3.06 | 1.17 |
| Silicon Dioxide | 0.10 | 0.04 |
| Magnesium Stearate | 1.04 | 0.40 |
| Total Core | 104.20 | 40.00 |
| Lactose Hydrate | 107.8 | 41.40 |
| Hypromellose 2208 | 46.9 | 18.00 |
| (100000 MPa · s) | | |
| Magnesium Stearate | 1.56 | 0.60 |
| Total Mantle | 156.40 | 66.70 |
| Total Tablet | 260.60 | 100.00 |

The tablets were made according to the following steps. The core tablets were prepared using a wet granulation process. The granulation batch size was 680 g. Drug was passed through the Quadro Comil U5 with an 813 micron screen at 2000 rpm. Hydroxypropyl cellulose was passed through a 600 micron mesh screen. Drug and hydroxypropyl cellulose were granulated with purified water using a Diosna P1/6 equipped with a 4 L bowl. The wet granules were screened through an 1180 micron mesh screen and dried on trays in an oven at 30° C. for 6 hours.

The core blend batch size was 30.0 g. The dried granules and the silicon dioxide were then passed through a 600 micron mesh screen, combined in a glass jar and blended in a Turbula mixer for 2 minutes. Magnesium stearate was passed through a 250 micron screen and added to the blend before blending an additional 1.5 minutes. Core tablets (104.2 mg) were compressed using a Manesty FlexiTab single station tablet press with ¼ inch (6.35 mm) round standard concave tooling at approximately 3 kN force. The core tablets had a final hardness of 6.2 to 7.0 kp (about 61 to 69 Newtons).

The mantle blend was prepared using a direct compression process and a batch size of 10 g. The hypromellose 2208 (100000 MPa·s) and lactose hydrate were passed through a 600 micron mesh screen, combined in a glass bottle and blended for 5 minutes on a Turbula mixer. Magnesium stearate was passed through a 250 micron screen and added to the blend and blended an additional 1.5 minutes. The mantle blend was then applied to the core tablets using the Carver Press with 5/16 inch (7.94 mm) round standard concave tooling. Half the mantle blend (78.2 mg) was weighed out, added to the die, and tamped slightly to flatten. Then, the core tablet was placed into the die and pressed down gently into the mantle blend. The second half of the mantle blend (78.2 mg) was then added on top of the core tablet and the mantle was compressed using 1.1 metric ton (MT) force. The final compression coated tablets had a total weight of 260.6 mg with a drug loading of 100 mg (38.37%) or 53.5 mg equivalents MMF. The tablets had a final hardness ranging from 13.1 to 14.0 kp (about 128 to 137 Newtons).

### Example 13

### Safety, Tolerability, and Pharmacokinetics of Example 8 Dosage Form

A randomized, double-blind crossover, food effect, single-dose study of the safety, tolerability, and pharmacokinetics of the oral dosage form of Example 8 in healthy adult subjects was conducted. Twelve healthy adult volunteers (males and females) participated in the study. All twelve subjects received a dosage form of Example 8, once in a fed condition and once in a fasted condition, with a two-week washout between treatments. The fasted dosing was achieved by dosing the subject following an overnight fast while the fed dosing was achieved by dosing the subject after consuming a high fat-content breakfast. The dosage form contained 100 mg drug, or 53.5 mg equivalents of MMF.

Blood samples were collected from all subjects prior to dosing, and at 0.5, 1, 1.5, 2, 3, 4, 5, 6, 7, 8, 10, 12, 16, 24, 30, 36, 48, 60, 72, 84, 96, 108 and 120 hours after dosing. Urine samples were collected from all subjects prior to dosing, and complete urine output was obtained at the 0-4, 4-8, 8-12, 12-24, 24-36, 36-48, 48-72, 72-96 and 96-120 hour intervals after dosing. Blood samples were quenched immediately with acetonitrile and frozen. Sample aliquots were prepared for analysis of (i) MMF, (ii) drug, and (iii) other potential metaboliltes using sensitive and specific LC/MS/MS methods.

The plasma molar concentrations of MMF and MMF-glutathione adducts following oral dosing of the formulation prepared according to Example 8 to fasted and fed healthy adult patients is shown in FIG. 5 and FIG. 6, respectively. Table 13 shows the mean (SD) pharmacokinetic data in fed and fasted patients. The “Mean AUC_{molar-MMF}” and “Mean AUC_{molar-MMF-GA}” values presented in the table are the average of the individual values for these parameters in each subject. Similarly, the “Mean AUC_{molar-MMF-GA}:AUC_{molar-MMF} Ratio (%)” presented in the table is the average of the individual ratio values calculated in each subject, and therefore is not identical to the ratio of the “Mean AUC_{molar-MMF}” and “Mean AUC_{molar-MMF-GA}” values.

TABLE 13
PK Data for Example 8 Dosage Form

| | | | | Mean |
|---|---|---|---|---|
| | | Mean | Mean | AUC_{molar-MMF-GA}:mean |
| | | AUC_{molar-MMF-GA} | AUC_{molar-MMF} | AUC_{molar-MMF} |
| N | Food | (μM · hr) | (μM · hr) | Ratio (%) |
| 12 | Fast- | 0.240 | 4.50 | 4.72 |
| | ed | | | |
| 12 | Fed | 0.696 | 5.67 | 12.4 |

MMF release from the formulation was sustained and minimally affected by food. The drug was well tolerated during the trial. All 12 subjects completed the dosing period. All adverse events were mild.

### Example 14

Delayed release tablets were made having the ingredients shown in Table 14:

TABLE 14
Composition of Enteric Coated Delayed Release Tablet

| | | | Quantity | Quantity |
|---|---|---|---|---|
| Component | Manufacturer | Role | (mg/tablet) | (% w/w) |
| Active Drug | XenoPort (Santa | MMF Source | 200.00 mg | 78.38 |
| | Clara, CA) | | 107 mg-eqs MMF | |
| Hydroxypropyl | Ashland (Hopewell, | Binder | 6.19 | 2.42 |
| Cellulose | VA) | | | |
| Lactose | Foremost | Filler | 38.28 | 15.00 |
| Monohydrate | (Rothschild, WI) | | | |
| Croscarmellose | FMC BioPolymer | Disintegrant | 7.66 | 3.00 |
| Sodium | (Philadelphia, PA) | | | |
| Silicon Dioxide | Cabot (Tuscola, IL) | Glidant | 0.51 | 0.20 |
| Magnesium | Mallinckrodt (St. | Lubricant | 2.55 | 1.00 |
| Stearate | Louis, MO) | | | |
| | | Total Core | 255.19 | 100.00 |
| Opadry | Colorcon (West | Barrier coat | 6.80 | 2.66 |
| 03O19184 | Point, PA) | | | |
| | | Total | 6.80 | 2.66 |
| | | Barrier | | |
| | | Coating | | |
| Methacrylic Acid | Evonik Industries | Enteric | 21.10 | 8.27 |
| Co-polymer | (Essen, Germany) | polymer | | |
| Dispersion | | | | |
| Triethyl Citrate | Vertellus | Plasticizer | 1.10 | 0.43 |
| | (Greensboro, NC) | | | |
| PlasACRYL ™ | Emerson Resources | Anti-tacking | 2.10 | 0.82 |
| T20 | (Norristown, PA) | agent | | |
| | | Total | 24.30 | 9.52 |
| | | Enteric | | |
| | | Coating | | |
| | | Total Tablet | 286.29 | 112.19 |

The tablets were made according to the following steps. The core tablets were prepared using a wet granulation process. The granulation was performed in two batches at 463.9 g per batch. Drug and hydroxypropyl cellulose were passed through a conical mill with a 610 micron round holed screen. Drug and hydroxypropyl cellulose were then combined in a Key KG-5 granulator bowl and mixed with water addition for approximately 9 minutes. The wet granules were dried in a Glatt GPCG-1 fluid bed dryer at 40° C. The two portions of dried granules were combined and blended with the silicon dioxide in an 8 quart (7.6 liter) V-blender for 5 minutes and then sized by passing through a conical mill with an approximately 1300 micron grater type screen. The milled granules were blended with the croscarmellose sodium and lactose monohydrate for 10 minutes in an 8 quart (7.6 l) V-blender. The magnesium stearate was passed through a 600 micron mesh screen and blended with the additional core materials in the V-blender for 5 minutes. Core tablets (254.87 mg) were compressed using a GlobePharma Minipress II rotary tablet press with 11/32 inch (8.7 mm) round concave tooling. The core tablets had a final mean hardness of approximately 15.5 kp. An aqueous suspension was prepared by mixing with an impeller 68.85 g Opadry 03O19184 with 792.0 g of purified water. The water contained in the suspension is removed during the film coating process and therefore not included in the final formulation in Table 14. The tablets were coated with the aqueous suspension in an O' Hara Technologies Labcoat M coater with a 12″ (30.5 cm) diameter perforated pan until the desired weight gain of barrier coat was achieved. The coating process occurred at an inlet temperature of approximately 52° C. and an outlet temperature of 37° C. After coating, the tablets were dried for 2 hours at 40° C. An aqueous suspension was prepared by mixing with an impeller 578.7 g methacrylic acid copolymer dispersion, 9.0 g triethyl citrate, 86.5 g PlasACRYL™ T20 with 325.8 g water. The water contained in (i) the methacrylic acid copolymer dispersion and (ii) the PlasACRYL™ T20 is removed during the film coating process and therefore not included in the final formulation in Table 14. The tablets were coated with the aqueous suspension in an O' Hara Technologies Labcoat M coater with a 12″ (30.5 cm) diameter perforated pan until the desired weight gain of enteric film was achieved. The coating process occurred at an inlet temperature of approximately 40° C. and an outlet temperature of 30° C. After coating, the tablets were dried for 2 hours at 40° C.

### Example 15

A randomized, double-blind crossover, food effect, single-dose study of the safety, tolerability, and pharmacokinetics of the oral dosage forms of Example 14 in healthy adult subjects was conducted. Twelve healthy adult volunteers (males and females) participated in the study. All twelve of the subjects received a dosage form of Example 14, once in a fed condition and once in a fasted condition, with a two-week washout between treatments. The fasted dosing was achieved by dosing the subject following an overnight fast while the fed dosing was achieved by dosing the subject after consuming a high fat-content breakfast. The tested dosage forms contained 200 mg of active drug, or 107 mg equivalents of methyl hydrogen fumarate.

Blood samples were collected from all subjects prior to dosing, and at 0.5, 1, 1.5, 2, 3, 4, 5, 6, 7, 8, 10, 12, 16, 24, 30, 36, 48, 60, 72, 84, 96, 108 and 120 hours after dosing. Urine samples were collected from all subjects prior to dosing, and complete urine output was obtained at the 0-4, 4-8, 8-12, 12-24, 24-36, 36-48, 48-72, 72-96 and 96-120 hour intervals after dosing. Blood samples were quenched immediately with acetonitrile and frozen. Sample aliquots were prepared for analysis of (i) methyl hydrogen fumarate, (ii) drug, and (iii) other potential metabolites using sensitive and specific LC/MS/MS methods.

The plasma concentration of MMF following oral dosing of the formulation prepared according to Example 14 to fasted and fed healthy adult patients is shown in FIG. 7 and FIG. 8, respectively. Table 15 shows the mean (SD) pharmacokinetic data for the Example 14 dosage forms in fed and fasted patients. The “Mean AUC_{molar-MMF}” and “Mean AUC_{molar-MMF-GA}” values presented in the table are the average of the individual values for these parameters in each subject. Similarly, the “Mean AUC_{molar-MMF-GA}:AUC_{molar-MMF} Ratio (%)” presented in the table is the average of the individual ratio values calculated in each subject, and therefore is not identical to the ratio of the “Mean AUC_{molar-MMF}” and “Mean AUC_{molar-MMF-GA}” values.

TABLE 15
PK Data for Example 14 Dosage Form

| | | | | Mean |
|---|---|---|---|---|
| | | Mean | Mean | AUC_{molar-MMF-GA}:mean |
| | | AUC_{molar-MMF-GA} | AUC_{molar-MMF} | AUC_{molar-MMF} |
| N | Food | (μM · hr) | (μM · hr) | Ratio (%) |
| 12 | Fast- | 0.533 | 5.84 | 9.20 |
| | ed | | | |
| 12 | Fed | 1.463 | 4.69 | 39.6 |

The drug was well tolerated during the trial. All 12 subjects completed the dosing period. All adverse events were mild.

The range of values of AUC_{molar-MMF-GA}:AUC_{molar-MMF} Ratio (%) disclosed herein have been shown to be associated with efficacy in animal models of MS and psoriasis. In the MOG35-55 mouse EAE model of MS, C57BL/6 mice (6 females) were injected subcutaneously with MOG35-55 peptide in CFA with Mycobacterium tuberculosis. Pertussis toxin (200 ng) was injected IV on Day 0 and Day 2 post-immunization. Animals received oral active drug (90 mg-eq MMF/kg twice daily) or vehicle on Days 3 to 29. Daily EAE clinical disease scores (5 point scale) were recorded. Blood levels of MMF and MMF-glutathione adducts were determined by LC/MS/MS. Active drug produced significant reduction in EAE clinical score (Day 29 and overall AUC) compared to vehicle. The AUC_{molar-MMF-GA}:AUC_{molar-MMF} Ratio (%) in mice dosed with Active drug at 90 mg/kg was 23.6%.

In the imiquimod (IMQ) mouse model of psoriasis. Balb/c mice (10 males/group) received daily topical IMQ (5% cream) on shaved back and right ear for 5 days. Animals received oral active drug (90 mg-eq MMF/kg twice daily) or vehicle from Day −5 to Day 5. Erythema score was the primary outcome measure. Active drug showed a significant reduction in erythema score versus control. The AUC_{molar-MMF-GA}:AUC_{molar-MMF} Ratio (%) in mice dosed with active drug at 90 mg/kg was 23.6%.

Finally, it should be noted that there are alternative ways of implementing the embodiments disclosed herein. Accordingly, the present embodiments are to be considered as illustrative and not restrictive, and the claims are not to be limited to the details given herein, but may be modified within the scope and equivalents thereof.

1. A method of administering a therapeutically effective amount of monomethyl fumarate to treat a disease in each patient of a population of patients in need of such treatment, comprising administering the monomethyl fumarate to each patient to achieve (i) a mean total area under a curve plotting average molar concentration of monomethyl fumarate-glutathione adducts in blood plasma of the patients versus time (mean AUC_{molar-MMF-GA}); and (ii) a mean total area under a curve plotting average molar concentration of monomethyl fumarate in the blood plasma of the patients versus time (mean AUC_{molar-MMF}); wherein a ratio of mean AUC_{molar-MMF-GA}:mean AUC_{molar-MMF} is greater than 2%.

2. The method of claim 1, wherein the ratio of mean AUC_{molar-MMF-GA}:mean AUC_{molar-MMF} is greater than 4%.

3. The method of claim 1, wherein the ratio of mean AUC_{molar-MMF-GA}:mean AUC_{molar-MMF} is from 4% to 100%.

4. The method of claim 1, wherein the ratio of mean AUC_{molar-MMF-GA}:mean AUC_{molar-MMF} is from 5% to 50%.

5. The method of claim 1, wherein the ratio of mean AUC_{molar-MMF-GA}:mean AUC_{molar-MMF} is from 5% to 20%.

6. The method of claim 1, wherein the ratio of mean AUC_{molar-MMF-GA}:mean AUC_{molar-MMF} is from 20% to 35%.

7. The method of claim 1, wherein the ratio of mean AUC_{molar-MMF-GA}:mean AUC_{molar-MMF} is from 35% to 50%.

8. The method of claim 1, wherein the monomethyl fumarate-glutathione adducts are chosen from: [chemical structure]
and diastereomers thereof.

9. The method of claim 1, wherein the monomethyl fumarate-glutathione adducts are chosen from: [chemical structure]
and diastereomers thereof.

10. The method of claim 1, wherein the monomethyl fumarate is administered to the patient at a dose of from 300 to 600 mg monomethyl fumarate per day.

11. The method of claim 1, wherein the monomethyl fumarate is administered to the patient at a dosing frequency of from once per day to three times per day.

12. The method of claim 1, wherein the disease is chosen from multiple sclerosis and psoriasis.

13. A method of administering a therapeutically effective amount of monomethyl fumarate to treat a disease in a patient in need of such treatment, comprising administering the monomethyl fumarate to the patient at a monomethyl fumarate dose and dosing frequency that achieves (i) a total area under a curve plotting average molar concentration of monomethyl fumarate-glutathione adducts in blood plasma of the patient versus time (AUC_{molar-MMF-GA}); and (ii) a total area under a curve plotting average molar concentration of monomethyl fumarate in the blood plasma of the patient versus time (AUC_{molar-MMF}); wherein a ratio of AUC_{molar-MMF-GA}:AUC_{molar-MMF} is greater than 2%.

14. The method of claim 13, wherein the ratio of AUC_{molar-MMF-GA}:AUC_{molar-MMF} is greater than 4%.

15. The method of claim 13, wherein the ratio of AUC_{molar-MMF-GA}:AUC_{molar-MMF} is from 4% to 100%.

16. The method of claim 13, wherein the ratio of AUC_{molar-MMF-GA}:AUC_{molar-MMF} is from 5% to 50%.

17. The method of claim 13, wherein the ratio of AUC_{molar-MMF-GA}:AUC_{molar-MMF} is from 5% to 20%.

18. The method of claim 13, wherein the ratio of AUC_{molar-MMF-GA}:AUC_{molar-MMF} is from 20% to 35%.

19. The method of claim 13, wherein the ratio of AUC_{molar-MMF-GA}:AUC_{molar-MMF} is from 35% to 50%.

20. The method of claim 13, wherein the monomethyl fumarate-glutathione adducts are chosen from: [chemical structure]
and diastereomers thereof.

21. The method of claim 13, wherein the monomethyl fumarate-glutathione adducts are chosen from: [chemical structure]
and diastereomers thereof.

22. The method of claim 13, wherein the monomethyl fumarate is administered to the patient at a dose of from 300 to 600 mg monomethyl fumarate per day.

23. The method of claim 13, wherein the monomethyl fumarate is administered to the patient at a dosing frequency of from once per day to three times per day.

24. The method of claim 13, wherein the disease is multiple sclerosis.

25. The method of claim 13, wherein the disease is chosen from psoriasis.

26. A method of administering a therapeutically effective amount of monomethyl fumarate to treat a disease in each patient of a population of patients in need of such treatment, comprising administering the monomethyl fumarate to the patient at a monomethyl fumarate dose and dosing frequency that achieves formation of MMF-GA (monomethylfumarate-glutathione adducts) in blood plasma; and wherein a mean maximum concentration (C_{max-MMF-GA}) of MMF-GA in the patients is at least 2% of a mean maximum concentration (C_{max-MMF}) of monomethyl fumarate in the patients.

27. The method of claim 26, wherein the mean maximum concentration (C_{max-MMF-GA}) of MMF-GA is at least 4% of the mean maximum concentration (C_{max-MMF}) of monomethyl fumarate.

28. The method of claim 26, wherein the mean maximum concentration (C_{max-MMF-GA}) of MMF-GA is from 4% to 50% of the mean maximum concentration (C_{max-MMF}) of monomethyl fumarate.

29. The method of claim 26, wherein the mean maximum concentration (C_{max-MMF-GA}) of MMF-GA is from 5% to 50% of the mean maximum concentration (C_{max-MMF}) of monomethyl fumarate.

30. The method of claim 26, wherein the mean maximum concentration (C_{max-MMF-GA}) of MMF-GA is from 5% to 20% of the mean maximum concentration (C_{max-MMF}) of monomethyl fumarate.

31. The method of claim 26, wherein the mean maximum concentration (C_{max-MMF-GA}) of MMF-GA is from 20% to 35% of the mean maximum concentration (C_{max-MMF}) of monomethyl fumarate.

32. The method of claim 26, wherein the mean maximum concentration (C_{max-MMF-GA}) of MMF-GA is from 35% to 50% of the mean maximum concentration (C_{max-MMF}) of monomethyl fumarate.

33. The method of claim 26, wherein the monomethyl fumarate-glutathione adducts are chosen from: [chemical structure]
and diastereomers thereof.

34. The method of claim 26, wherein the monomethyl fumarate-glutathione adducts are chosen from: [chemical structure]
and diastereomers thereof.

35. The method of claim 26, wherein the monomethyl fumarate is administered to the patient at a dose of from 300 to 600 mg monomethyl fumarate per day.

36. The method of claim 26, wherein the monomethyl fumarate is administered to the patient at a dosing frequency of from once per day to three times per day.

37. The method of claim 26, wherein the disease is chosen from multiple sclerosis and psoriasis.

38. The method of claim 26, wherein the MMF-GA concentration in the blood plasma reaches the C_{max-MMF-GA} value within a time period of 2 to 10 hours after the administration.

39. A method of administering a therapeutically effective amount of monomethyl fumarate to treat a disease in a patient in need of such treatment, comprising administering the monomethyl fumarate to the patient at a monomethyl fumarate dose and dosing frequency that achieves formation of MMF-GA (monomethylfumarate-glutathione adducts) in blood plasma; and wherein maximum concentration (C_{max-MMF-GA}) of MMF-GA in the patient is at least 2% of a maximum concentration (C_{max-MMF}) of monomethyl fumarate in the patient.

40. The method of claim 39, wherein the maximum concentration (C_{max-MMF-GA}) of MMF-GA is at least 4% of the maximum concentration (C_{max-MMF}) of monomethyl fumarate.

41. The method of claim 39, wherein the maximum concentration (C_{max-MMF-GA}) of MMF-GA is from 4% to 50% of the maximum concentration (C_{max-MMF}) of monomethyl fumarate.

42. The method of claim 39, wherein the maximum concentration (C_{max-MMF-GA}) of MMF-GA is from 5% to 50% of the maximum concentration (C_{max-MMF}) of monomethyl fumarate.

43. The method of claim 39, wherein the maximum concentration (C_{max-MMF-GA}) of MMF-GA is from 5% to 20% of the maximum concentration (C_{max-MMF}) of monomethyl fumarate.

44. The method of claim 39, wherein the maximum concentration (C_{max-MMF-GA}) of MMF-GA is from 20% to 35% of the maximum concentration (C_{max-MMF}) of monomethyl fumarate.

45. The method of claim 39, wherein the maximum concentration (C_{max-MMF-GA}) of MMF-GA is from 35% to 50% of the maximum concentration (C_{max-MMF}) of monomethyl fumarate.

46. The method of claim 39, wherein the monomethyl fumarate-glutathione adducts are chosen from: [chemical structure]
and diastereomers thereof.

47. The method of claim 39, wherein the monomethyl fumarate-glutathione adducts are chosen from: [chemical structure]
and diastereomers thereof.

48. The method of claim 39, wherein the monomethyl fumarate is administered to the patient at a dose of from 300 to 600 mg monomethyl fumarate per day.

49. The method of claim 39, wherein the monomethyl fumarate is administered to the patient at a dosing frequency of from once per day to three times per day.

50. The method of claim 39, wherein the disease is chosen from multiple sclerosis and psoriasis.

51. The method of claim 39, wherein the MMF-GA concentration in the blood plasma reaches the C_{max-MMF-GA} value within a time period of 2 to 10 hours after the administration.

52. The method of claim 8, wherein the monomethyl fumarate-glutathione adducts are in ionic forms.

53. The method of claim 8, wherein the monomethyl fumarate-glutathione adducts are in zwitterionic forms.

54. The method of claim 8, wherein the monomethyl fumarate-glutathione adducts are chosen from [chemical structure]
and diastereomers thereof.

55. The method of claim 8, wherein the monomethyl fumarate-glutathione adducts are chosen from [chemical structure]
and diastereomers thereof.

56. The method of claim 8, wherein the monomethyl fumarate-glutathione adduct form is a result of a physiological transformation.

57. The method of claim 8, wherein the monomethyl fumarate-glutathione adducts are in any naturally occurring physiological salt form.

58. The method of claim 8, wherein the monomethyl fumarate-glutathione adducts are in the form of an HCl salt or a phosphate salt.

59. The method of claim 1, wherein the administration is systemic administration.

60. The method of claim 1, wherein the administration is oral administration.
